(19) United States
(12) Patent Application Publication
(10) Pub. No.: US 2023/0322659 A1
(43) Pub. Date: Oct. 12, 2023

(54) STROBILURIN TYPE COMPOUNDS AND THEIR USE FOR COMBATING PHYTOPATHOGENIC FUNGI

(71) Applicant: BASF SE, Ludwigshafen (DE)

(72) Inventors: Sarang Kulkarni, Pune (IN); Chandan Dey, Navi Mumbai (IN); Manojkumar Poonoth, Navi Mumbai (IN); Rakesh Rath, Navi Mumbai (IN); Ronan La Vezouet, Ludwigshafen (DE); Smriti Khanna, Navi Mumbai (IN); Christian Harald Winter, Ludwigshafen (DE); Marcus Fehr, Limburgerhof (DE); Andreas Koch, Limburgerhof (DE); Wassilios Grammenos, Ludwigshafen (DE); Vanessa Tegge, Limburgerhof (DE)

(21) Appl. No.: 18/015,361

(22) PCT Filed: Jul. 6, 2021
(86) PCT No.: PCT/EP2021/068645

§ 371 Date: Jan. 10, 2023

(30) Foreign Application Priority Data
Jul. 16, 2020 (EP) 20186146.5

(51) Int. Cl. C07C 251/60; A01P 3/00; C07C 251/58; A01N 37/50

(52) U.S. Cl. CPC C07C 251/60; A01N 37/50; C07C 251/58; A01P 3/00

(57) ABSTRACT

The present invention relates to the use of strobilurin type compounds of formula (I) and the N-oxides and the salts thereof for combating phytopathogenic fungi containing an amino acid substitution F129L in the mitochondrial cytochrome b protein (also referred to as F129L mutation in the mitochondrial cytochrome b gene) conferring resistance to Qo inhibitors, and to methods for combating such fungi. The invention also relates to novel compounds, processes for preparing these compounds, to compositions comprising at least one such compound, and to seeds coated with at least one such compound.

The present invention relates the use of strobilurin type compounds of formula I and the N-oxides and the salts thereof for combating phytopathogenic fungi containing an amino acid substitution F129L in the mitochondrial cytochrome b protein (also referred to as F129L mutation in the mitochondrial cytochrome b gene) conferring resistance to Qo inhibitors (QoI), and to methods for combating such fungi. The invention also relates to novel compounds, processes for preparing these compounds, to compositions comprising at least one such compound, to plant health applications, and to seeds coated with at least one such compound. The present invention also relates to a method for controlling soybean rust fungi (Phakopsora pachyrhizi) with the amino acid substitution F129L in the mitochondrial cytochrome b protein.

“Qo inhibitor,” as used herein, includes any substance that is capable of diminishing and/or inhibiting respiration by binding to a ubihydroquinone oxidation center of a cytochrome bc₁ complex in mitochondria. The oxidation center is typically located on the outer side of the inner mitochondrial membrane. Many of these compounds are also known as strobilurin-type or strobilurin analogue compounds.

The mutation F129L in the mitochondrial cytochrome b (CYTB) gene shall mean any substitution of nucleotides of codon 129 encoding “F” (phenylalanine; e.g. TTT or TTC) that leads to a codon encoding “L” (leucine; e.g. TTA, TTG, TTG, CTT, CTC, CTA or CTG), for example the substitution of the first nucleotide of codon 129 ‘T’ to ‘C’ (TTT to CTT), in the CYTB (cytochrome b) gene resulting in a single amino acid substitution in the position 129 from F to L in the cyto-chrome b protein. Such F129L mutation is known to confer resistance to Qo inhibitors

QoI fungicides, often referred to as strobilurin-type fungicides (Sauter 2007: Chapter 13.2. Strobilurins and other complex III inhibitors. In: Krämer, W.; Schirmer, U. (Ed.)—Modern Crop Protection Compounds. Volume 2. Wiley-VCH Verlag 457-495), are conventionally used to control a number of fungal pathogens in crops. Qo inhibitors typically work by inhibiting respiration by binding to a ubihydroquinone oxidation center of a cytochrome bc₁ complex (electron transport complex III) in mitochondria. Said oxidation center is located on the outer side of the inner mitochondrial membrane. A prime example of the use of QoIs includes the use of, for example, strobilurins on wheat for the control of Septoria tritici (also known as Mycosphaerella graminicola), which is the cause of wheat leaf blotch. Unfortunately, widespread use of such QoIs has resulted in the selection of mutant pathogens which are resistant to such QoIs (Gisi et al., Pest Manag Sci 56, 833-841, (2000)). Resistance to QoIs has been detected in several phytopathogenic fungi such as Blumeria graminis, Mycosphaerella fijiensis, Pseudoperonspora cubensis or Venturia inaequalis. The major part of resistance to QoIs in agricultural uses has been attributed to pathogens containing a single amino acid residue substitution G143A in the cytochrome b gene for their cytochrome bc₁ complex, the target protein of QoIs which have been found to be controlled by specific QoIs (WO 2013/092224). Despite several commercial QoI fungicides have also been widely used in soybean rust control, the single amino acid residue substitution G143A in the cytochrome b protein conferring resistance to QoI fungicides was not observed.

Instead soybean rust acquired a different genetic mutation in the cytochrome b gene causing a single amino acid substitution F129L which also confers resistance against QoI fungicides. The efficacy of QoI fungicides used against soybean rust conventionally, i.e. pyraclostrobin, azoxystrobin, picoxystrobin, orysastrobin, dimoxystrobin and metominostrobin, has decreased to a level with practical problems for agricultural practice (e.g. Klosowski et al (2016) Pest Manag Sci 72, 1211-1215).

Although it seems that trifloxystrobin was less affected by the F129L mutation to the same degree as other QoI fungicides such as azoxystrobin and pyraclostrobin, trifloxystrobin was never as efficacious on a fungal population bearing the F129L QoI resistance mutation as on a sensitive population (Crop Protection 27, (2008) 427-435).

Thus, new methods are desirable for controlling pathogen induced diseases in crops comprising plants subjected to pathogens containing an amino acid substitution F129L in the mitochondrial cytochrome b protein conferring resistance to Qo inhibitors. Furthermore, in many cases, in particular at low application rates, the fungicidal activity of the known fungicidal strobilurin compounds is unsatisfactory, especially in case that a high proportion of the fungal pathogens contain an amino acid substitution F129L in the mitochondrial cytochrome b protein conferring resistance to Qo inhibitors. Besides there is an ongoing need for new fungicidally active compounds which are more effective, less toxic and/or environmentally safer. Based on this, it was also an object of the present invention to provide compounds having improved activity and/or a broader activity spectrum against phytopathogenic fungi and/or even further reduced toxicity against non target organisms such as vertebrates and invertebrates.

Certain strobilurin type compounds have been inter alia described in EP 370629, EP 463488 and WO 98/23156. However, it is not mentioned that these compounds inhibit fungal pathogens containing a F129L substitution in the mitochondrial cytochrome b protein conferring resistance to Qo inhibitors.

The strobilurin-analogue compounds according to the present invention differ from those described in the abovementioned publications inter alia by bearing the specific group R³ as defined herein and/or by lacking the methyl group bound to the oxime linker in the side chain.

Accordingly, the invention provides novel compounds of formula I

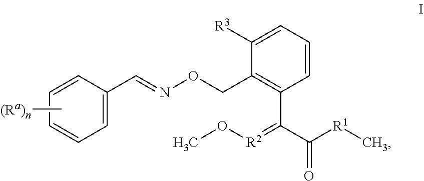

wherein
R¹ is selected from O and NH;
R² is selected from CH and N;
R³ is selected from halogen, CN, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, —O—C₁-C₄-alkyl, —O—C₁-C₄-haloalkyl, —O—C₃-C₆-cycloalkyl, —C₁-C₂-alkyl-C₃-C₆-cycloalkyl, phenyl, 3- to 6-membered heterocyclo-alkyl and 5- or 6-membered heteroaryl,
   wherein said heterocycloalkyl and heteroaryl besides carbon atoms contain 1, 2 or 3 heteroatoms selected from N, O and S, provided that such heterocycloalkyl and heteroaryl cannot contain 2 contiguous atoms selected from O and S,
   wherein said phenyl, heterocycloalkyl and heteroaryl are bound directly or via an oxygen atom or via a C₁-C₂-alkylene linker, and wherein said phenyl and heteroaryl are unsubstituted or substituted by 1, 2 or 3 identical or different substituents selected from halogen, CN, NH₂, NO₂, C₁-C₄-alkyl, C₁-C₄-haloalkyl, —O—C₁-C₄-alkyl and —O—C₁-C₄-haloalkyl;
R^{a} is selected from halogen, CN, —NR⁵R⁶, C₁-C₄-alkyl, C₂-C₄-alkenyl, C₂-C₄-alkynyl, —O—C₁-C₄-alkyl, —C(═N—O—C₁-C₄-alkyl)-C₁-C₄-alkyl, —C(═O)—C₁-C₄-alkyl, —O—CH₂—C(═N—O—C₁-C₁-C₄-alkyl)-C₁-C₄-alkyl, C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 6-membered heterocyclo-alkyl, 3- to 6-membered heterocycloalkenyl and 5- or 6-membered heteroaryl, wherein said heterocycloalkyl, heterocycloalkenyl and heteroaryl besides carbon atoms contain 1, 2 or 3 heteroatoms selected from N, O and S, provided that such heterocycloalkyl, heterocycloalkenyl and heteroaryl cannot contain 2 contiguous atoms selected from O and S,
   wherein said phenyl, heterocycloalkyl, heterocycloalkenyl and heteroaryl are bound directly or via an oxygen atom or via a C₁-C₂-alkylene linker,
   and wherein the aliphatic and cyclic moieties of R^{a} are unsubstituted or carry 1, 2, 3, 4 or up to the maximum number of identical or different groups R^{b}:
   R^{b} is selected from halogen, CN, NH₂, NO₂, C₁-C₄-alkyl, C₁-C₄-haloalkyl, —O—C₁-C₄-alkyl and —O—C₁-C₄-haloalkyl;
   R⁵, R⁶ are independently of each other selected from the group consisting of H, C₁-C₆-alkyl, C₁-C₆-haloalkyl and C₂-C₄-alkynyl;
n is an integer selected from 0, 1, 2, 3, 4 and 5;
   and in form or stereoisomers and tautomers thereof, and the N-oxides and the agriculturally acceptable salts thereof.

Although the present invention will be described with respect to particular embodiments, this description is not to be construed in a limiting sense.

Before describing in detail exemplary embodiments of the present invention, definitions important for understanding the present invention are given. As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

The term “compounds I” refers to compounds of formula I. Likewise, this terminology applies to all sub-formulae, e. g. “compounds I.2” refers to compounds of formula I.2 or “compounds V” refers to compounds of formula V, etc.

The term “independently” when used in the context of selection of substituents for a variable, it means that where more than one substituent is selected from a number of possible substituents, those substituents may be the same or different.

The organic moieties or groups mentioned in the above definitions of the variables are collective terms for individual listings of the individual group members. The term “Cᵥ-C_{w}” indicates the number of carbon atom possible in each case.

The term “halogen” refers to fluorine, chlorine, bromine and iodine.

The term “C₁-C₄-alkyl” refers to a straight-chained or branched saturated hydrocarbon group having 1 to 4 carbon atoms, for example, methyl (CH₃), ethyl (C₂H₅), propyl, 1-methylethyl (isopropyl), butyl, 1-methylpropyl, 2-methylpropyl, 1,1-dimethylethyl.

The term “C₂-C₄-alkenyl” refers to a straight-chain or branched unsaturated hydrocarbon radical having 2 to 4 carbon atoms and a double bond in any position such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₄-alkynyl” refers to a straight-chain or branched unsaturated hydrocarbon radical having 2 to 4 carbon atoms and containing at least one triple bond such as ethynyl, prop-1-ynyl, prop-2-ynyl, but-1-ynyl, but-2-ynyl, but-3-ynyl, 1-methyl-prop-2-ynyl.

The term “C₁-C₄-haloalkyl” refers to a straight-chained or branched alkyl group having 1 to 4 carbon atoms wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂-C₂F₅, CF₂-C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “—O—C₁-C₄-alkyl” refers to a straight-chain or branched alkyl group having 1 to 4 carbon atoms which is bonded via an oxygen, at any position in the alkyl group, e.g. OCH₃, OCH₂CH₃, O(CH₂)₂CH₃, 1-methylethoxy, O(CH₂)₃CH₃, 1-methyl¬propoxy, 2-methylpropoxy or 1,1-dimethylethoxy.

The term “C₃-C₆-cycloalkyl” refers to monocyclic saturated hydrocarbon radicals having 3 to 6 carbon ring members, such as cyclopropyl (C₃H₅), cyclobutyl, cyclopentyl or cyclohexyl. The term “C₃-C₆-cycloalkenyl” refers to monocyclic saturated hydrocarbon radicals having 3 to 6 carbon ring members and one or more double bonds.

The term “3- to 6-membered heterocycloalkyl” refers to 3- to 6-membered monocyclic saturated ring system having besides carbon atoms one or more heteroatoms, such as O, N, S as ring members. The term “C₃-C₆-membered heterocycloalkenyl” refers to 3- to 6-membered monocyclic ring system having besides carbon atoms one or more heteroatoms, such as O, N and S as ring members, and one or more double bonds.

The term “—C₁-C₄-alkyl-C₃-C₆-cycloalkyl” refers to alkyl having 1 to 4 carbon atoms (as defined above), wherein one hydrogen atom of the alkyl radical is replaced by a cycloalkyl radical having 3 to 6 carbon atoms.

The term “phenyl” refers to C₆H₅.

The term “5- or 6-membered heteroaryl” which contains 1, 2, 3 or 4 heteroatoms from the group consisting of O, N and S, is to be understood as meaning aromatic heterocycles having 5 or 6 ring atoms. Examples include:
5-membered heteroaryl which in addition to carbon atoms, e.g. contain 1, 2 or 3 N atoms and/or one sulfur and/or one oxygen atom: for example 2-thienyl, 3-thienyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which, in addition to carbon atoms, e.g. contain 1, 2, 3 or 4 N atoms as ring members, e.g. 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

The term “C₁-C₂-alkylene linker” means a divalent alkyl group such as —CH₂— or —CH₂—CH₂— that is bound at one end to the core structure of formula I and at the other end to the particular substituent.

As used herein, the “compounds”, in particular “compounds I” include all the stereoisomeric and tautomeric forms and mixtures thereof in all ratios, prodrugs, isotopic forms, their agriculturally acceptable salts, N-oxides and S-oxides thereof.

The term “stereoisomer” is a general term used for all isomers of individual compounds that differ only in the orientation of their atoms in space. The term stereoisomer includes mirror image isomers (enantiomers), mixtures of mirror image isomers (racemates, racemic mixtures), geometric (cis/trans or E/Z) isomers, and isomers of compounds with more than one chiral center that are not mirror images of one another (diastereoisomers). The term “tautomer” refers to the coexistence of two (or more) compounds that differ from each other only in the position of one (or more) mobile atoms and in electron distribution, for example, keto-enol tautomers. The term “agriculturally acceptable salts” as used herein, includes salts of the active compounds which are prepared with acids or bases, depending on the particular substituents found on the compounds described herein. “N-oxide” refers to the oxide of the nitrogen atom of a nitrogen-containing heteroaryl or heterocycle. N-oxide can be formed in the presence of an oxidizing agent for example peroxide such as m-chloro-perbenzoic acid or hydrogen peroxide. N-oxide refers to an amine oxide, also known as amine-N-oxide, and is a chemical compound that contains N→O bond.

In respect of the variables, the embodiments of the intermediates correspond to the embodiments of the compounds I.

Preference is given to those compounds I and where applicable also to compounds of all sub-formulae provided herein, e. g. formulae I.1 and I.2, and to the intermediates such as compounds II, III, IV and V, wherein the substituents and variables (such as n, R¹, R², R³, R⁵, R⁶, R^{a}, and R^{b}) have independently of each other or more preferably in combination (any possible combination of 2 or more substituents as defined herein) the following meanings:

Preference is also given to the uses, methods, mixtures and compositions, wherein the definitions (such as phytopathogenic fungi, treatments, crops, compounds II, further active ingredients, solvents, solid carriers) have independently of each other or more preferably in combination the following meanings and even more preferably in combination (any possible combination of 2 or more definitions as provided herein) with the preferred meanings of compound I herein:

One embodiment of the invention relates to the abovementioned use and or method of application (herein collectively referred to as “use”) of compounds I, wherein R¹ is selected from O and NH; and R² is selected from CH and N, provided that R² is N in case R¹ is NH. More preferably R¹ is NH. In particular, R¹ is NH and R² is N.

According to another embodiment, R³ is selected from halogen, CN, C₁-C₄-alkyl, C₂-C₄-alkenyl, C₁-C₄-haloalkyl, C₂-C₄-haloalkenyl, C₃-C₆-cycloalkyl, —O—C₁-C₄-alkyl, —O—C₁-C₄-haloalkyl, —C₁-C₂-alkyl-C₃-C₆-cycloalkyl and 3- to 6-membered heterocycloalkyl; more preferably from halogen, C₁-C₂-alkyl, C₂-alkenyl, C₁-C₂-haloalkyl, —O—C₁-C₂-alkyl, —O—C₁-C₂-haloalkyl, C₃-C₄-cycloalkyl, —C₁-C₂-alkyl-C₃-C₄-cycloalkyl and 3- to 4-membered heterocycloalkyl; even more preferably from C₁-C₂-alkyl, C₁-C₂-haloalkyl, C₃-C₄-cycloalkyl, —O—C₁-C₂-alkyl and —O—C₁-C₂-haloalkyl; particularly preferred from methyl and C₁-C₂-haloalkyl, in particular methyl.

According to a further embodiment, n is 1, 2, 3, 4 or 5; more preferably n is 1, 2 or 3, even more preferably n is 1 or 2; in particular n is 1.

According to a further embodiment, n is 0, 1, 2 or 3, more preferably 0, 1 or 2, in particular 0.

According to a further embodiment, R^{a} is selected from CN, C₁-C₄-alkyl, C₂-C₄-alkenyl, C₂-C₄-alkynyl, —O—C₁-C₄-alkyl, —C(═O)—C₁-C₄-alkyl, —C(═N—O—C₁-C₄-alkyl)-C₁-C₄-alkyl, —O—CH₂—(═N—O—C₁-C₄-alkyl)-C₁-C₄-alkyl, —C(═N—O—C₁-C₄-alkyl)-C(═O—NH—C₁-C₄-alkyl), C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 5-membered heterocycloalkyl, 3- to 5-membered heterocycloalkenyl and 5- or 6-membered heteroaryl, wherein said heterocycloalkyl, hetercycloalkenyl and heteroaryl besides carbon atoms contain 1, 2 or 3 heteroatoms selected from N, O and S, provided that such heterocycloalkyl, heterocycloalkenyl and heteroaryl cannot contain 2 contiguous atoms selected from O and S, wherein said phenyl, heterocycloalkyl, hetercycloalkenyl and heteroaryl are bound directly or via an oxygen atom or via a C₁-C₂-alkylene linker, and wherein the aliphatic and cyclic moieties of R^{a} are unsubstituted or carry 1, 2, or 3 of identical or different groups R^{b} which independently of one another are selected from halogen, CN, NH₂, NO₂, C₁-C₂-alkyl and C₁-C₂-haloalkyl.

More preferably, R^{a} is selected from CN, C₁-C₄-alkyl, C₂-C₄-alkenyl, C₂-C₄-alkynyl, —O—C₁-C₄-alkyl, —C(═O)—C₁-C₂-alkyl, —C(═N—O—C₁-C₂-alkyl)-C₁-C₂-alkyl, —O—CH₂—C(═N—O—C₁-C₂-alkyl)-C₁-C₂-alkyl, —C(═N—O—C₁-C₂-alkyl)-C(═O—NH—C₁-C₂-alkyl), C₃-C₄-cycloalkyl, C₃-C₄-cycloalkenyl, —C₁-C₂-alkyl-C₃-C₄-cycloalkyl, —O—C₃-C₄-cycloalkyl, phenyl, 3- to 5-membered heterocycloalkyl and 5- or 6-membered heteroaryl, wherein said heterocycloalkyl and heteroaryl besides carbon atoms contain 1 or 2 heteroatoms selected from N, O and S, provided that such heterocycloalkyl and heteroaryl cannot contain 2 contiguous atoms selected from O and S, wherein said phenyl, heterocycloalkyl and heteroaryl are bound directly or via an oxygen atom or via a methylene linker, and wherein the aliphatic or cyclic moieties of R^{a} are unsubstituted or carry 1, 2, or 3 of identical or different groups R^{b} which independently of one another are selected from halogen, CN, C₁-C₂-alkyl and C₁-C₂-haloalkyl.

Even more preferably R^{a} is selected from C₁-C₃-alkyl, C₂-C₃-alkenyl, C₂-C₃-alkynyl, —O—C₁-C₃-alkyl, —C(═O)—C₁-C₂-alkyl, —C(═N—O—C₁-C₂-alkyl)-C₁-C₂-alkyl, C₃-C₄-cycloalkyl, —C₁-C₂-alkyl-C₃-C₄-cycloalkyl, —O—C₃-C₄-cycloalkyl, phenyl, 3- to 5-membered heterocycloalkyl and 5- or 6-membered heteroaryl, wherein said heterocycloalkyl and heteroaryl besides carbon atoms contain 1 or 2 heteroatoms selected from N, O and S, provided that such heterocycloalkyl and heteroaryl cannot contain 2 contiguous atoms selected from O and S, wherein said phenyl and heteroaryl are bound directly or via an oxygen atom or via a methylene linker, and wherein the aliphatic and cyclic moieties of R^{a} are unsubstituted or carry 1, 2 or 3 of identical or different groups R^{b} which independently of one another are selected from halogen, CN, methyl and C₁-haloalkyl.

Particularly preferred R^{a} are selected from halogen, C₁-C₄-alkyl, C₂-C₃-alkenyl, C₂-C₃-alkynyl, —O—C₁-C₄-alkyl, —C(═N—O—C₁-C₂-alkyl)-C₁-C₂-alkyl and phenyl, wherein the aliphatic or cyclic moieties of R^{a} are unsubstituted or carry 1, 2 or 3 of identical or different groups R^{b} which independently of one another are selected from halogen, CN, methyl and C₁-haloalkyl.

According to a further embodiment, R⁵, R⁶ are independently of each other preferably selected from the group consisting of H, C₁-C₄-alkyl, C₁-C₄-haloalkyl and C₂-C₄-alkynyl, more preferably from H and C₁-C₄-alkyl.

According to a further preferred embodiment, the present invention relates to compounds of formula I wherein:
R¹ is selected from O and NH; and
R² is selected from CH and N, provided that R² is N in case R¹ is NH;
R³ is selected from halogen, CN, C₁-C₄-alkyl, C₁-C₄-haloalkyl and C₃-C₄-cycloalkyl;
R^{a} is selected from halogen, CN, —NR⁵R⁶, C₁-C₄-alkyl, C₂-C₄-alkenyl, C₂-C₄-alkynyl, —O—C₁-C₄-alkyl, —C(═N—O—C₁-C₄-alkyl)-C₁-C₄-alkyl, —C(═O)—C₁-C₄-alkyl, —O—CH₂—C(═N—O—C₁-C₄-alkyl)-C₁-C₄-alkyl, C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 6-membered heterocycloalkyl, 3- to 6-membered heterocycloalkenyl and 5- or 6-membered heteroaryl, wherein said heterocycloalkyl, heterocycloalkenyl and heteroaryl besides carbon atoms contain 1, 2 or 3 heteroatoms selected from N, O and S, provided that such heterocycloalkyl, heterocycloalkenyl and heteroaryl cannot contain 2 contiguous atoms selected from O and S,
   wherein said phenyl, heterocycloalkyl, heterocycloalkenyl and heteroaryl are bound directly or via an oxygen atom or via a C₁-C₂-alkylene linker,
   and wherein the aliphatic and cyclic moieties of R^{a} are unsubstituted or carry 1, 2, 3, 4 or up to the maximum number of identical or different groups R^{b}:
   R^{b} is selected from halogen, CN, NH₂, NO₂, C₁-C₄-alkyl, C₁-C₄-haloalkyl, —O—C₁-C₄-alkyl and —O—C₁-C₄-haloalkyl;
   R⁵, R⁶ are independently of each other selected from the group consisting of H, C₁-C₆-alkyl and C₂-C₄-alkynyl;
n is an integer selected from 0, 1, 2 and 3;
   and in form or stereoisomers and tautomers thereof, and the N-oxides and the agriculturally acceptable salts thereof.

According to a further preferred embodiment, the invention provides novel compounds of formula I, wherein:
R¹ is selected from O and NH;
R² is N;
R³ is selected from C₁-C₄-alkyl and C₁-C₄-haloalkyl, ;
R^{a} is selected from halogen, C₁-C₄-alkyl and —O—C₁-C₄-alkyl, and wherein the aliphatic moieties of R^{a} are unsubstituted or carry 1, 2 or 3 identical or different groups R^{b}:
   R^{b} is selected from halogen;
n is an integer selected from 0, 1, 2, 3, 4 and 5;
   and in form or stereoisomers and tautomers thereof, and the N-oxides and the agriculturally acceptable salts thereof.

According to a further embodiment, R¹ is NH, R² is N and R³ is methyl, which compounds are of formula I.1:

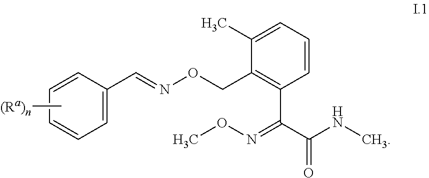

According to a further embodiment, R¹ is O, R² is N and R³ is methyl, which compounds are of formula I.2:

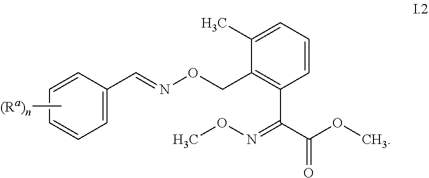

According to a further embodiment, R¹ is O, R² is CH and R³ is methyl, which compounds are of formula I.3:

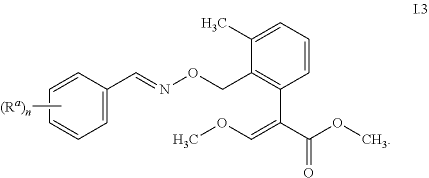

Preferably, R³ of compounds I is one of the following radicals 3-1 to 3-8:

| No. | R³ |
|---|---|
| 3-1 | CH₃ |
| 3-2 | OCH₃ |
| 3-3 | CHF₂ |
| 3-4 | C₃H₅ |
| 3-5 | CH═CH₂ |
| 3-6 | CH₂CH═C(CH₃)₂ |
| 3-7 | CF₃ |
| 3-8 | C(═NOCH₃)CH₃ |

Even more preferably R³ is CH₃, OCH₃, CF₃, CHF₂ or C₃H₅, in particular CH₃.

Particularly preferred embodiments of the invention relate to compounds I, wherein the R^{a} is selected of one of the following radicals a-1 to a-17:

| No. | R^{a} |
|---|---|
| a-1 | F |
| a-2 | Cl |
| a-3 | Br |
| a-4 | CH₃ |
| a-5 | CHF₂ |
| a-6 | CF₃ |
| a-7 | OCH₃ |
| a-8 | OCHF₂ |
| a-9 | OCF₃ |
| a-1 | C₂H₅ |
| a-11 | CH₂CF₃ |
| a-12 | CH═CH₂ |
| a-13 | C₆H₅ |
| a-14 | C≡CH |
| a-15 | C≡CCH₃ |
| a-16 | C₃H₅ |
| a-17 | C(═NOCH₃)CH₃ |

According to a further embodiment, n is 1. More preferably, R^{a} is in ortho-position (2-R^{a}), which compounds are of formula I.A:

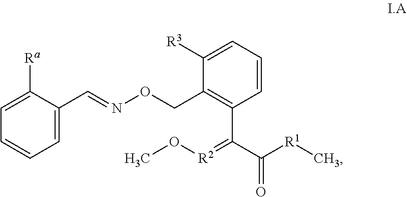

wherein even more preferably R² is N. According to a further embodiment, R^{a} is in meta-position (3-R^{a}), which compounds are of formula I.B:

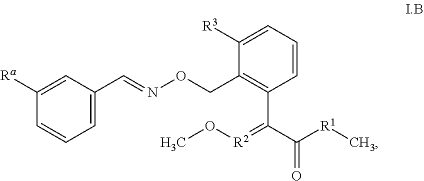

wherein even more preferably R² is N.

According to a further embodiment, n is 2. More preferably, n is 2 and the two R^{a} substituents are both in meta -position (3,5-R^{a}), which compounds are of formula I.C:

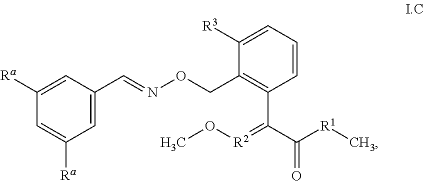

wherein even more preferably R² is N. According to a further embodiment, n is 2 and the two R^{a} substituents are both in ortho-position (2,6-R^{a}), which compounds are of formula I.D:

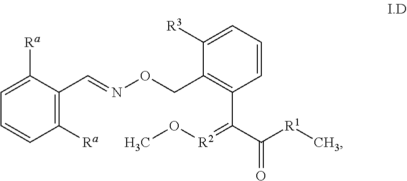

wherein even more preferably R² is N. According to a further embodiment, n is 2 and the two R^{a} substituents are in ortho- and meta-position, which compounds are of formula I.E:

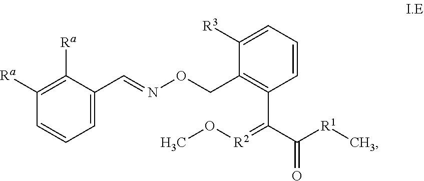

wherein even more preferably R² is N. According to a further embodiment, n is 2 and the two R^{a} substituents are in ortho- and para-position, which compounds are of formula I.F:

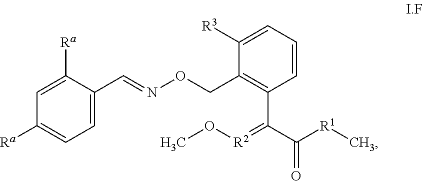

wherein even more preferably R² is N.

In an embodiment, compounds I are of formula I.1 and n and R^{a}, are as per any row of Table A below, which compounds are named I.1-A-1 to I.1-A-809.

In another embodiment, compounds I are of formula I.2 and n and R^{a} are as per any row of Table A below, which compounds are named I.2-A-1 to I.2-A-809.
In another embodiment, compounds I are of formula I.3 and n and R^{a} are as per any row of Table A below, which compounds are named I.3-A-1 to I.3-A-809.

TABLE A

| No. | n | R^{a} |
|---|---|---|
| A-1 | 0 | — |
| A-2 | 1 | 2-F |
| A-3 | 1 | 2-Cl |
| A-4 | 1 | 2-Br |
| A-5 | 1 | 2-CH₃ |
| A-6 | 1 | 2-CHF₂ |
| A-7 | 1 | 2-CF₃ |
| A-8 | 1 | 2-OCH₃ |
| A-9 | 1 | 2-OCHF₂ |
| A-10 | 1 | 2-OCF₃ |
| A-11 | 1 | 2-C₂H₅ |
| A-12 | 1 | 2-CH₂CF₃ |
| A-13 | 1 | 2-CH═CH₂ |
| A-14 | 1 | 2-C₆H₅ |
| A-15 | 1 | 2-C≡CH |
| A-16 | 1 | 2-C≡CCH₃ |
| A-17 | 1 | 2-C₃H₅ |
| A-18 | 1 | 2-C(═NOCH₃)CH₃ |
| A-19 | 1 | 2-CN |
| A-20 | 1 | 3-F |
| A-21 | 1 | 3-Cl |
| A-22 | 1 | 3-Br |
| A-23 | 1 | 3-CH₃ |
| A-24 | 1 | 3-CHF₂ |
| A-25 | 1 | 3-CF₃ |
| A-26 | 1 | 3-OCH₃ |
| A-27 | 1 | 3-OCHF₂ |
| A-28 | 1 | 3-OCF₃ |
| A-29 | 1 | 3-C₂H₅ |
| A-30 | 1 | 3-CH₂CF₃ |
| A-31 | 1 | 3-CH═CH₂ |
| A-32 | 1 | 3-C₆H₅ |
| A-33 | 1 | 3-C≡CH |
| A-34 | 1 | 3-C≡CCH₃ |
| A-35 | 1 | 3-C₃H₅ |
| A-36 | 1 | 3-C(═NOCH₃)CH₃ |
| A-37 | 1 | 3-CN |
| A-38 | 1 | 4-F |
| A-39 | 1 | 4-Cl |
| A-40 | 1 | 4-Br |
| A-41 | 1 | 4-CH₃ |
| A-42 | 1 | 4-CHF₂ |
| A-43 | 1 | 4-CF₃ |
| A-44 | 1 | 4-OCH₃ |
| A-45 | 1 | 4-OCHF₂ |
| A-46 | 1 | 4-OCF₃ |
| A-47 | 1 | 4-C₂H₅ |
| A-48 | 1 | 4-CH₂CF₃ |
| A-49 | 1 | 4-CH═CH₂ |
| A-50 | 1 | 4-C₆H₅ |
| A-51 | 1 | 4-C≡CH |
| A-52 | 1 | 4-C≡CCH₃ |
| A-53 | 1 | 4-C₃H₅ |
| A-54 | 1 | 4-C(═NOCH₃)CH₃ |
| A-55 | 1 | 4-CN |
| A-56 | 0 | - |
| A-57 | 1 | 2-F |
| A-58 | 1 | 2-Cl |
| A-59 | 1 | 2-Br |
| A-60 | 1 | 2-CH₃ |
| A-61 | 1 | 2-CHF₂ |
| A-62 | 1 | 2-CF₃ |
| A-63 | 1 | 2-OCH₃ |
| A-64 | 1 | 2-OCHF₂ |
| A-65 | 1 | 2-OCF₃ |
| A-66 | 1 | 2-C₂H₅ |
| A-67 | 1 | 2-CH₂CF₃ |
| A-68 | 1 | 2-CH═CH₂ |
| A-69 | 1 | 2-C₆H₅ |
| A-70 | 1 | 2-C≡CH |
| A-71 | 1 | 2-C≡CCH₃ |
| A-72 | 1 | 2-C₃H₅ |
| A-73 | 1 | 2-C(═NOCH₃)CH₃ |
| A-74 | 1 | 2-CN |
| A-75 | 1 | 3-F |
| A-76 | 1 | 3-Cl |
| A-77 | 1 | 3-Br |
| A-78 | 1 | 3-CH₃ |
| A-79 | 1 | 3-CHF₂ |
| A-80 | 1 | 3-CF₃ |
| A-81 | 1 | 3-OCH₃ |
| A-82 | 1 | 3-OCHF₂ |
| A-83 | 1 | 3-OCF₃ |
| A-84 | 1 | 3-C₂H₅ |
| A-85 | 1 | 3-CH₂CF₃ |
| A-86 | 1 | 3-CH═CH₂ |
| A-87 | 1 | 3-C₆H₅ |
| A-88 | 1 | 3-C≡CH |
| A-89 | 1 | 3-C≡CCH₃ |
| A-90 | 1 | 3-C₃H5 |
| A-91 | 1 | 3-C(═NOCH₃)CH₃ |
| A-92 | 1 | 3-CN |
| A-93 | 1 | 4-F |
| A-94 | 1 | 4-Cl |
| A-95 | 1 | 4-Br |
| A-96 | 1 | 4-CH₃ |
| A-97 | 1 | 4-CHF₂ |
| A-98 | 1 | 4-CF₃ |
| A-99 | 1 | 4-OCH₃ |
| A-100 | 1 | 4-OCHF₂ |
| A-101 | 1 | 4-OCF₃ |
| A-102 | 1 | 4-C₂H₅ |
| A-103 | 1 | 4-CH₂CF₃ |
| A-104 | 1 | 4-CH═CH₂ |
| A-105 | 1 | 4-C₆H₅ |
| A-106 | 1 | 4-C≡CH |
| A-107 | 1 | 4-C≡CCH₃ |
| A-108 | 1 | 4-C₃H₅ |
| A-109 | 1 | 4-C(═NOCH₃)CH₃ |
| A-110 | 1 | 4-CN |
| A-111 | 0 | — |
| A-112 | 1 | 2-F |
| A-113 | 1 | 2-Cl |
| A-114 | 1 | 2-Br |
| A-115 | 1 | 2-CH₃ |
| A-116 | 1 | 2-CHF₂ |
| A-117 | 1 | 2-CF₃ |
| A-118 | 1 | 2-OCH₃ |
| A-119 | 1 | 2-OCHF₂ |
| A-120 | 1 | 2-OCF₃ |
| A-121 | 1 | 2-C₂H₅ |
| A-122 | 1 | 2-CH₂CF₃ |
| A-123 | 1 | 2-CH═CH₂ |
| A-124 | 1 | 2-C₆H₅ |
| A-125 | 1 | 2-C≡CH |
| A-126 | 1 | 2-C≡CCH₃ |
| A-127 | 1 | 2-C₃H₅ |
| A-128 | 1 | 2-C(═NOCH₃)CH₃ |
| A-129 | 1 | 2-CN |
| A-130 | 1 | 3-F |
| A-131 | 1 | 3-Cl |
| A-132 | 1 | 3-Br |
| A-133 | 1 | 3-CH₃ |
| A-134 | 1 | 3-CHF₂ |
| A-135 | 1 | 3-CF₃ |
| A-136 | 1 | 3-OCH₃ |
| A-137 | 1 | 3-OCHF₂ |
| A-138 | 1 | 3-OCF₃ |
| A-139 | 1 | 3-C₂H₅ |
| A-140 | 1 | 3-CH₂CF₃ |
| A-141 | 1 | 3-CH═CH₂ |
| A-142 | 1 | 3-C₆H₅ |
| A-143 | 1 | 3-C≡CH |
| A-144 | 1 | 3-C≡CCH₃ |
| A-145 | 1 | 3-C₃H₅ |
| A-146 | 1 | 3-C(═NOCH₃)CH₃ |
| A-147 | 1 | 3-CN |
| A-148 | 1 | 4-F |
| A-149 | 1 | 4-Cl |
| A-150 | 1 | 4-Br |
| A-151 | 1 | 4-CH₃ |
| A-152 | 1 | 4-CHF₂ |
| A-153 | 1 | 4-CF₃ |
| A-154 | 1 | 4-OCH₃ |
| A-155 | 1 | 4-OCHF₂ |
| A-156 | 1 | 4-OCF₃ |
| A-157 | 1 | 4-C₂H₅ |
| A-158 | 1 | 4-CH₂CF₃ |
| A-159 | 1 | 4-CH═CH₂ |
| A-160 | 1 | 4-C₆H₅ |
| A-161 | 1 | 4-C≡CH |
| A-162 | 1 | 4-C≡CCH₃ |
| A-163 | 1 | 4-C₃H₅ |
| A-164 | 1 | 4-C(═NOCH₃)CH₃ |
| A-165 | 1 | 4-CN |
| A-166 | 0 | — |
| A-167 | 1 | 2-F |
| A-168 | 1 | 2-Cl |
| A-169 | 1 | 2-Br |
| A-170 | 1 | 2-CH₃ |
| A-171 | 1 | 2-CHF₂ |
| A-172 | 1 | 2-CF₃ |
| A-173 | 1 | 2-OCH₃ |
| A-174 | 1 | 2-OCHF₂ |
| A-175 | 1 | 2-OCF₃ |
| A-176 | 1 | 2-C₂H₅ |
| A-177 | 1 | 2-CH₂CF₃ |
| A-178 | 1 | 2-CH═CH₂ |
| A-179 | 1 | 2-C₆H₅ |
| A-180 | 1 | 2-C≡CH |
| A-181 | 1 | 2-C≡CCH₃ |
| A-182 | 1 | 2-C₃H₅ |
| A-183 | 1 | 2-C(═NOCH₃)CH₃ |
| A-184 | 1 | 2-CN |
| A-185 | 1 | 3-F |
| A-186 | 1 | 3-Cl |
| A-187 | 1 | 3-Br |
| A-188 | 1 | 3-CH₃ |
| A-189 | 1 | 3-CHF₂ |
| A-190 | 1 | 3-CF₃ |
| A-191 | 1 | 3-OCH₃ |
| A-192 | 1 | 3-OCHF₂ |
| A-193 | 1 | 3-OCF₃ |
| A-194 | 1 | 3-C₂H₅ |
| A-195 | 1 | 3-CH₂CF₃ |
| A-196 | 1 | 3-CH═CH₂ |
| A-197 | 1 | 3-C₆H₅ |
| A-198 | 1 | 3-C≡CH |
| A-199 | 1 | 3-C≡CCH₃ |
| A-200 | 1 | 3-C₃H₅ |
| A-201 | 1 | 3-C(═NOCH₃)CH₃ |
| A-202 | 1 | 3-CN |
| A-203 | 1 | 4-F |
| A-204 | 1 | 4-Cl |
| A-205 | 1 | 4-Br |
| A-206 | 1 | 4-CH₃ |
| A-207 | 1 | 4-CHF₂ |
| A-208 | 1 | 4-CF₃ |
| A-209 | 1 | 4-OCH₃ |
| A-210 | 1 | 4-OCHF₂ |
| A-211 | 1 | 4-OCF₃ |
| A-212 | 1 | 4-C₂H₅ |
| A-213 | 1 | 4-CH₂CF₃ |
| A-214 | 1 | 4-CH═CH₂ |
| A-215 | 1 | 4-C₆H₅ |
| A-216 | 1 | 4-C≡CH |
| A-217 | 1 | 4-C≡CCH₃ |
| A-218 | 1 | 4-C₃H₅ |
| A-219 | 1 | 4-C(═NOCH₃)CH₃ |
| A-220 | 1 | 4-CN |
| A-221 | 0 | — |
| A-222 | 1 | 2-F |
| A-223 | 1 | 2-Cl |
| A-224 | 1 | 2-Br |
| A-225 | 1 | 2-CH₃ |
| A-226 | 1 | 2-CHF₂ |
| A-227 | 1 | 2-CF₃ |
| A-228 | 1 | 2-OCH₃ |
| A-229 | 1 | 2-OCHF₂ |
| A-230 | 1 | 2-OCF₃ |
| A-231 | 1 | 2-C₂H₅ |
| A-232 | 1 | 2-CH₂CF₃ |
| A-233 | 1 | 2-CH═CH₂ |
| A-234 | 1 | 2-C₆H₅ |
| A-235 | 1 | 2-C≡CH |
| A-236 | 1 | 2-C≡CCH₃ |
| A-237 | 1 | 2-C₃H₅ |
| A-238 | 1 | 2-C(═NOCH₃)CH₃ |
| A-239 | 1 | 2-CN |
| A-240 | 1 | 3-F |
| A-241 | 1 | 3-Cl |
| A-242 | 1 | 3-Br |
| A-243 | 1 | 3-CH₃ |
| A-244 | 1 | 3-CHF₂ |
| A-245 | 1 | 3-CF₃ |
| A-246 | 1 | 3-OCH₃ |
| A-247 | 1 | 3-OCHF₂ |
| A-248 | 1 | 3-OCF₃ |
| A-249 | 1 | 3-C₂H₅ |
| A-250 | 1 | 3-CH₂CF₃ |
| A-251 | 1 | 3-CH═CH₂ |
| A-252 | 1 | 3-C₆H₅ |
| A-253 | 1 | 3-C≡CH |
| A-254 | 1 | 3-C≡CCH₃ |
| A-255 | 1 | 3-C₃H₅ |
| A-256 | 1 | 3-C(═NOCH₃)CH₃ |
| A-257 | 1 | 3-CN |
| A-258 | 1 | 4-F |
| A-259 | 1 | 4-Cl |
| A-260 | 1 | 4-Br |
| A-261 | 1 | 4-CH₃ |
| A-262 | 1 | 4-CHF₂ |
| A-263 | 1 | 4-CF₃ |
| A-264 | 1 | 4-OCH₃ |
| A-265 | 1 | 4-OCHF₂ |
| A-266 | 1 | 4-OCF₃ |
| A-267 | 1 | 4-C₂H₅ |
| A-268 | 1 | 4-CH₂CF₃ |
| A-269 | 1 | 4-CH═CH₂ |
| A-270 | 1 | 4-C₆H₅ |
| A-271 | 1 | 4-C≡CH |
| A-272 | 1 | 4-C≡CCH₃ |
| A-273 | 1 | 4-C₃H₅ |
| A-274 | 1 | 4-C(═NOCH₃)CH₃ |
| A-275 | 1 | 4-CN |
| A-276 | 0 | — |
| A-277 | 1 | 2-F |
| A-278 | 1 | 2-Cl |
| A-279 | 1 | 2-Br |
| A-280 | 1 | 2-CH₃ |
| A-281 | 1 | 2-CHF₂ |
| A-282 | 1 | 2-CF₃ |
| A-283 | 1 | 2-OCH₃ |
| A-284 | 1 | 2-OCHF₂ |
| A-285 | 1 | 2-OCF₃ |
| A-286 | 1 | 2-C₂H₅ |
| A-287 | 1 | 2-CH₂CF₃ |
| A-288 | 1 | 2-CH═CH₂ |
| A-289 | 1 | 2-C₆H₅ |
| A-290 | 1 | 2-C≡CH |
| A-291 | 1 | 2-C≡CCH₃ |
| A-292 | 1 | 2-C₃H₅ |
| A-293 | 1 | 2-C(═NOCH₃)CH₃ |
| A-294 | 1 | 2-CN |
| A-295 | 1 | 3-F |
| A-296 | 1 | 3-Cl |
| A-297 | 1 | 3-Br |
| A-298 | 1 | 3-CH₃ |
| A-299 | 1 | 3-CHF₂ |
| A-300 | 1 | 3-CF₃ |
| A-301 | 1 | 3-OCH₃ |
| A-302 | 1 | 3-OCHF₂ |
| A-303 | 1 | 3-OCF₃ |
| A-304 | 1 | 3-C₂H₅ |
| A-305 | 1 | 3-CH₂CF₃ |
| A-306 | 1 | 3-CH═CH₂ |
| A-307 | 1 | 3-C₆H₅ |
| A-308 | 1 | 3-C≡CH |
| A-309 | 1 | 3-C≡CCH₃ |
| A-310 | 1 | 3-C₃H₅ |
| A-311 | 1 | 3-C(═NOCH₃)CH₃ |
| A-312 | 1 | 3-CN |
| A-313 | 1 | 4-F |
| A-314 | 1 | 4-Cl |
| A-315 | 1 | 4-Br |
| A-316 | 1 | 4-CH₃ |
| A-317 | 1 | 4-CHF₂ |
| A-318 | 1 | 4-CF₃ |
| A-319 | 1 | 4-OCH₃ |
| A-320 | 1 | 4-OCHF₂ |
| A-321 | 1 | 4-OCF₃ |
| A-322 | 1 | 4-C₂H₅ |
| A-323 | 1 | 4-CH₂CF₃ |
| A-324 | 1 | 4-CH═CH₂ |
| A-325 | 1 | 4-C₆H₅ |
| A-326 | 1 | 4-C≡CH |
| A-327 | 1 | 4-C≡CCH₃ |
| A-328 | 1 | 4-C₃H₅ |
| A-329 | 1 | 4-C(═NOCH₃)CH₃ |
| A-330 | 1 | 4-CN |
| A-331 | 0 | — |
| A-332 | 1 | 2-F |
| A-333 | 1 | 2-Cl |
| A-334 | 1 | 2-Br |
| A-335 | 1 | 2-CH₃ |
| A-336 | 1 | 2-CHF₂ |
| A-337 | 1 | 2-CF₃ |
| A-338 | 1 | 2-OCH₃ |
| A-339 | 1 | 2-OCHF₂ |
| A-340 | 1 | 2-OCF₃ |
| A-341 | 1 | 2-C₂H₅ |
| A-342 | 1 | 2-CH₂CF₃ |
| A-343 | 1 | 2-CH═CH₂ |
| A-344 | 1 | 2-C₆H₅ |
| A-345 | 1 | 2-C≡CH |
| A-346 | 1 | 2-C≡CCH₃ |
| A-347 | 1 | 2-C₃H₅ |
| A-348 | 1 | 2-C(═NOCH₃)CH₃ |
| A-349 | 1 | 2-CN |
| A-350 | 1 | 3-F |
| A-351 | 1 | 3-Cl |
| A-352 | 1 | 3-Br |
| A-353 | 1 | 3-CH₃ |
| A-354 | 1 | 3-CHF₂ |
| A-355 | 1 | 3-CF₃ |
| A-356 | 1 | 3-OCH₃ |
| A-357 | 1 | 3-OCHF₂ |
| A-358 | 1 | 3-OCF₃ |
| A-359 | 1 | 3-C₂H₅ |
| A-360 | 1 | 3-CH₂CF₃ |
| A-361 | 1 | 3-CH═CH₂ |
| A-362 | 1 | 3-C₆H₅ |
| A-363 | 1 | 3-C≡CH |
| A-364 | 1 | 3-C≡CCH₃ |
| A-365 | 1 | 3-C₃H₅ |
| A-366 | 1 | 3-C(═NOCH₃)CH₃ |
| A-367 | 1 | 3-CN |
| A-368 | 1 | 4-F |
| A-369 | 1 | 4-Cl |
| A-370 | 1 | 4-Br |
| A-371 | 1 | 4-CH₃ |
| A-372 | 1 | 4-CHF₂ |
| A-373 | 1 | 4-CF₃ |
| A-374 | 1 | 4-OCH₃ |
| A-375 | 1 | 4-OCHF₂ |
| A-376 | 1 | 4-OCF₃ |
| A-377 | 1 | 4-C₂H₅ |
| A-378 | 1 | 4-CH₂CF₃ |
| A-379 | 1 | 4-CH═CH₂ |
| A-380 | 1 | 4-C₆H₅ |
| A-381 | 1 | 4-C≡CH |
| A-382 | 1 | 4-C≡CCH₃ |
| A-383 | 1 | 4-C₃H₅ |
| A-384 | 1 | 4-C(═NOCH₃)CH₃ |
| A-385 | 1 | 4-CN |
| A-386 | 2 | 2-F, 3-F |
| A-387 | 2 | 2-F, 3-Cl |
| A-388 | 2 | 2-F, 3-Br |
| A-389 | 2 | 2-F, 3-CH₃ |
| A-390 | 2 | 2-F, 3-CHF₂ |
| A-391 | 2 | 2-F, 3-CF₃ |
| A-392 | 2 | 2-F, 3-OCH₃ |
| A-393 | 2 | 2-F, 3-OCHF₂ |
| A-394 | 2 | 2-F, 3-OCF₃ |
| A-395 | 2 | 2-F, 3-CH₂OCH₃ |
| A-396 | 2 | 2-F, 3-C₂H₅ |
| A-397 | 2 | 2-F, 3-CH₂CF₃ |
| A-398 | 2 | 2-F, 3-CH═CH₂ |
| A-399 | 2 | 2-F, 3-C≡CH |
| A-400 | 2 | 2-F, 3-C≡CCH₃ |
| A-401 | 2 | 2-F, 3-C₃H₅ |
| A-402 | 2 | 2-F, 3-C(═NOCH₃)CH₃ |
| A-403 | 2 | 2-F, 3-CN |
| A-404 | 2 | 2-F, 4-F |
| A-405 | 2 | 2-F, 4-Cl |
| A-406 | 2 | 2-F, 4-Br |
| A-407 | 2 | 2-F, 4-CH₃ |
| A-408 | 2 | 2-F, 4-CHF₂ |
| A-409 | 2 | 2-F, 4-CF₃ |
| A-410 | 2 | 2-F, 4-OCH₃ |
| A-411 | 2 | 2-F, 4-OCHF₂ |
| A-412 | 2 | 2-F, 4-OCF₃ |
| A-413 | 2 | 2-F, 4-CH₂OCH₃ |
| A-414 | 2 | 2-F, 4-C₂H₅ |
| A-415 | 2 | 2-F, 4-CH₂CF₃ |
| A-416 | 2 | 2-F, 4-CH═CH₂ |
| A-417 | 2 | 2-F, 4-C≡CH |
| A-418 | 2 | 2-F, 4-C≡CCH₃ |
| A-419 | 2 | 2-F, 4-C₃H₅ |
| A-420 | 2 | 2-F, 4-C(═NOCH₃)CH₃ |
| A-421 | 2 | 2-F, 4-CN |
| A-422 | 2 | 2-F, 6-F |
| A-423 | 2 | 2-F, 6-Cl |
| A-424 | 2 | 2-F, 6-Br |
| A-425 | 2 | 2-F, 6-CH₃ |
| A-426 | 2 | 2-F, 6-CHF₂ |
| A-427 | 2 | 2-F, 6-CF₃ |
| A-428 | 2 | 2-F, 6-OCH₃ |
| A-429 | 2 | 2-F, 6-OCHF₂ |
| A-430 | 2 | 2-F, 6-OCF₃ |
| A-431 | 2 | 2-F, 6-CH₂OCH₃ |
| A-432 | 2 | 2-F, 6-C₂H₅ |
| A-433 | 2 | 2-F, 6-CH₂CF₃ |
| A-434 | 2 | 2-F, 6-CH═CH₂ |
| A-435 | 2 | 2-F, 6-C≡CH |
| A-436 | 2 | 2-F, 6-C≡CCH₃ |
| A-437 | 2 | 2-F, 6-C₃H₅ |
| A-438 | 2 | 2-F, 6-C(═NOCH₃)CH₃ |
| A-439 | 2 | 2-Cl, 3-F |
| A-440 | 2 | 2-Cl, 3-Cl |
| A-441 | 2 | 2-Cl, 3-Br |
| A-442 | 2 | 2-Cl, 3-CH₃ |
| A-443 | 2 | 2-Cl, 3-CHF₂ |
| A-444 | 2 | 2-Cl, 3-CF₃ |
| A-445 | 2 | 2-Cl, 3-OCH₃ |
| A-446 | 2 | 2-Cl, 3-OCHF₂ |
| A-447 | 2 | 2-Cl, 3-OCF₃ |
| A-448 | 2 | 2-Cl, 3-CH₂OCH₃ |
| A-449 | 2 | 2-Cl, 3-C₂H₅ |
| A-450 | 2 | 2-Cl, 3-CH₂CF₃ |
| A-451 | 2 | 2-Cl, 3-CH═CH₂ |
| A-452 | 2 | 2-Cl, 3-C≡CH |
| A-453 | 2 | 2-Cl, 3-C≡CCH₃ |
| A-454 | 2 | 2-Cl, 3-C₃H₅ |
| A-455 | 2 | 2-Cl, 3-C(═NOCH₃)CH₃ |
| A-456 | 2 | 2-Cl, 3-CN |
| A-457 | 2 | 2-Cl, 4-F |
| A-458 | 2 | 2-Cl, 4-Cl |
| A-459 | 2 | 2-Cl, 4-Br |
| A-460 | 2 | 2-Cl, 4-CH₃ |
| A-461 | 2 | 2-Cl, 4-CHF₂ |
| A-462 | 2 | 2-Cl, 4-CF₃ |
| A-463 | 2 | 2-Cl, 4-OCH₃ |
| A-464 | 2 | 2-Cl, 4-OCHF₂ |
| A-465 | 2 | 2-Cl, 4-OCF₃ |
| A-466 | 2 | 2-Cl, 4-CH₂OCH₃ |
| A-467 | 2 | 2-Cl, 4-C₂H₅ |
| A-468 | 2 | 2-Cl, 4-CH₂CF₃ |
| A-469 | 2 | 2-Cl, 4-CH═CH₂ |
| A-470 | 2 | 2-Cl, 4-C≡CH |
| A-471 | 2 | 2-Cl, 4-C≡CCH₃ |
| A-472 | 2 | 2-Cl, 4-C₃H₅ |
| A-473 | 2 | 2-Cl, 4-C(═NOCH₃)CH₃ |
| A-474 | 2 | 2-Cl, 4-CN |
| A-475 | 2 | 2-Cl, 6-F |
| A-476 | 2 | 2-Cl, 6-Cl |
| A-477 | 2 | 2-Cl, 6-Br |
| A-478 | 2 | 2-Cl, 6-CH₃ |
| A-479 | 2 | 2-Cl, 6-CHF₂ |
| A-480 | 2 | 2-Cl, 6-CF₃ |
| A-481 | 2 | 2-Cl, 6-OCH₃ |
| A-482 | 2 | 2-Cl, 6-OCHF₂ |
| A-483 | 2 | 2-Cl, 6-OCF₃ |
| A-484 | 2 | 2-Cl, 6-CH₂OCH₃ |
| A-485 | 2 | 2-Cl, 6-C₂H₅ |
| A-486 | 2 | 2-Cl, 6-CH₂CF₃ |
| A-487 | 2 | 2-Cl, 6-CH═CH₂ |
| A-488 | 2 | 2-Cl, 6-C≡CH |
| A-489 | 2 | 2-Cl, 6-C≡CCH₃ |
| A-490 | 2 | 2-Cl, 6-C₃H₅ |
| A-491 | 2 | 2-Cl, 6-C(═NOCH₃)CH₃ |
| A-492 | 2 | 2-Br, 3-F |
| A-493 | 2 | 2-Br, 3-Cl |
| A-494 | 2 | 2-Br, 3-Br |
| A-495 | 2 | 2-Br, 3-CH₃ |
| A-496 | 2 | 2-Br, 3-CHF₂ |
| A-497 | 2 | 2-Br, 3-CF₃ |
| A-498 | 2 | 2-Br, 3-OCH₃ |
| A-499 | 2 | 2-Br, 3-OCHF₂ |
| A-500 | 2 | 2-Br, 3-OCF₃ |
| A-501 | 2 | 2-Br, 3-CH₂OCH₃ |
| A-502 | 2 | 2-Br, 3-C₂H₅ |
| A-503 | 2 | 2-Br, 3-CH₂CF₃ |
| A-504 | 2 | 2-Br, 3-CH═CH₂ |
| A-505 | 2 | 2-Br, 3-C≡CH |
| A-506 | 2 | 2-Br, 3-C≡CCH₃ |
| A-507 | 2 | 2-Br, 3-C₃H₅ |
| A-508 | 2 | 2-Br, 3-C(═NOCH₃)CH₃ |
| A-509 | 2 | 2-Br, 3-CN |
| A-510 | 2 | 2-Br, 4-F |
| A-511 | 2 | 2-Br, 4-Cl |
| A-512 | 2 | 2-Br, 4-Br |
| A-513 | 2 | 2-Br, 4-CH₃ |
| A-514 | 2 | 2-Br, 4-CHF₂ |
| A-515 | 2 | 2-Br, 4-CF₃ |
| A-516 | 2 | 2-Br, 4-OCH₃ |
| A-517 | 2 | 2-Br, 4-OCHF₂ |
| A-518 | 2 | 2-Br, 4-OCF₃ |
| A-519 | 2 | 2-Br, 4-CH₂OCH₃ |
| A-520 | 2 | 2-Br, 4-C₂H₅ |
| A-521 | 2 | 2-Br, 4-CH₂CF₃ |
| A-522 | 2 | 2-Br, 4-CH═CH₂ |
| A-523 | 2 | 2-Br, 4-C≡CH |
| A-524 | 2 | 2-Br, 4-C≡CCH₃ |
| A-525 | 2 | 2-Br, 4-C₃H₅ |
| A-526 | 2 | 2-Br, 4-C(═NOCH₃)CH₃ |
| A-527 | 2 | 2-Br, 4-CN |
| A-528 | 2 | 2-Br, 6-F |
| A-529 | 2 | 2-Br, 6-Cl |
| A-530 | 2 | 2-Br, 6-Br |
| A-531 | 2 | 2-Br, 6-CH₃ |
| A-532 | 2 | 2-Br, 6-CHF₂ |
| A-533 | 2 | 2-Br, 6-CF₃ |
| A-534 | 2 | 2-Br, 6-OCH₃ |
| A-535 | 2 | 2-Br, 6-OCHF₂ |
| A-536 | 2 | 2-Br, 6-OCF₃ |
| A-537 | 2 | 2-Br, 6-CH₂OCH₃ |
| A-538 | 2 | 2-Br, 6-C₂H₅ |
| A-539 | 2 | 2-Br, 6-CH₂CF₃ |
| A-540 | 2 | 2-Br, 6-CH═CH₂ |
| A-541 | 2 | 2-Br, 6-C≡CH |
| A-542 | 2 | 2-Br, 6-C≡CCH₃ |
| A-543 | 2 | 2-Br, 6-C₃H₅ |
| A-544 | 2 | 2-Br, 6-C(═NOCH₃)CH₃ |
| A-545 | 2 | 2-CH₃, 3-F |
| A-546 | 2 | 2-CH₃, 3-Cl |
| A-547 | 2 | 2-CH₃, 3-Br |
| A-548 | 2 | 2-CH₃, 3-CH₃ |
| A-549 | 2 | 2-CH₃, 3-CHF₂ |
| A-550 | 2 | 2-CH₃, 3-CF₃ |
| A-551 | 2 | 2-CH₃, 3-OCH₃ |
| A-552 | 2 | 2-CH₃, 3-OCHF₂ |
| A-553 | 2 | 2-CH₃, 3-OCF₃ |
| A-554 | 2 | 2-CH₃, 3-CH₂OCH₃ |
| A-555 | 2 | 2-CH₃, 3-C₂H₅ |
| A-556 | 2 | 2-CH₃, 3-CH₂CF₃ |
| A-557 | 2 | 2-CH₃, 3-CH═CH₂ |
| A-558 | 2 | 2-CH₃, 3-C≡CH |
| A-559 | 2 | 2-CH₃, 3-C≡CCH₃ |
| A-560 | 2 | 2-CH₃, 3-C₃H₅ |
| A-561 | 2 | 2-CH₃, 3-C(═NOCH₃)CH₃ |
| A-562 | 2 | 2-CH₃, 3-CN |
| A-563 | 2 | 2-CH₃, 4-F |
| A-564 | 2 | 2-CH₃, 4-Cl |
| A-565 | 2 | 2-CH₃, 4-Br |
| A-566 | 2 | 2-CH₃, 4-CH₃ |
| A-567 | 2 | 2-CH₃, 4-CHF₂ |
| A-568 | 2 | 2-CH₃, 4-CF₃ |
| A-569 | 2 | 2-CH₃, 4-OCH₃ |
| A-570 | 2 | 2-CH₃, 4-OCHF₂ |
| A-571 | 2 | 2-CH₃, 4-OCF₃ |
| A-572 | 2 | 2-CH₃, 4-CH₂OCH₃ |
| A-573 | 2 | 2-CH₃, 4-C₂H₅ |
| A-574 | 2 | 2-CH₃, 4-CH₂CF₃ |
| A-575 | 2 | 2-CH₃, 4-CH═CH₂ |
| A-576 | 2 | 2-CH₃, 4-C≡CH |
| A-577 | 2 | 2-CH₃, 4-C≡CCH₃ |
| A-578 | 2 | 2-CH₃, 4-C₃H₅ |
| A-579 | 2 | 2-CH₃, 4-C(═NOCH₃)CH₃ |
| A-580 | 2 | 2-CH₃, 4-CN |
| A-581 | 2 | 2-CH₃, 6-F |
| A-582 | 2 | 2-CH₃, 6-Cl |
| A-583 | 2 | 2-CH₃, 6-Br |
| A-584 | 2 | 2-CH₃, 6-CH₃ |
| A-585 | 2 | 2-CH₃, 6-CHF₂ |
| A-586 | 2 | 2-CH₃, 6-CF₃ |
| A-587 | 2 | 2-CH₃, 6-OCH₃ |
| A-588 | 2 | 2-CH₃, 6-OCHF₂ |
| A-589 | 2 | 2-CH₃, 6-OCF₃ |
| A-590 | 2 | 2-CH₃, 6-CH₂OCH₃ |
| A-591 | 2 | 2-CH₃, 6-C₂H₅ |
| A-592 | 2 | 2-CH₃, 6-CH₂CF₃ |
| A-593 | 2 | 2-CH₃, 6-CH═CH₂ |
| A-594 | 2 | 2-CH₃, 6-C≡CH |
| A-595 | 2 | 2-CH₃, 6-C≡CCH₃ |
| A-596 | 2 | 2-CH₃, 6-C₃H₅ |
| A-597 | 2 | 2-CH₃, 6-C(═NOCH₃)CH₃ |
| A-598 | 2 | 2-CHF₂, 3-F |
| A-599 | 2 | 2-CHF₂, 3-Cl |
| A-600 | 2 | 2-CHF₂, 3-Br |
| A-601 | 2 | 2-CHF₂, 3-CH₃ |
| A-602 | 2 | 2-CHF₂, 3-CHF₂ |
| A-603 | 2 | 2-CHF₂, 3-CF₃ |
| A-604 | 2 | 2-CHF₂, 3-OCH₃ |
| A-605 | 2 | 2-CHF₂, 3-OCHF₂ |
| A-606 | 2 | 2-CHF₂, 3-OCF₃ |
| A-607 | 2 | 2-CHF₂, 3-CH₂OCH₃ |
| A-608 | 2 | 2-CHF₂, 3-C₂H₅ |
| A-609 | 2 | 2-CHF₂, 3-CH₂CF₃ |
| A-610 | 2 | 2-CHF₂, 3-CH═CH₂ |
| A-611 | 2 | 2-CHF₂, 3-C≡CH |
| A-612 | 2 | 2-CHF₂, 3-C≡CCH₃ |
| A-613 | 2 | 2-CHF₂, 3-C₃H₅ |
| A-614 | 2 | 2-CHF₂, 3-C(═NOCH₃)CH₃ |
| A-615 | 2 | 2-CHF₂, 3-CN |
| A-616 | 2 | 2-CHF₂, 4-F |
| A-617 | 2 | 2-CHF₂, 4-Cl |
| A-618 | 2 | 2-CHF₂, 4-Br |
| A-619 | 2 | 2-CHF₂, 4-CH₃ |
| A-620 | 2 | 2-CHF₂, 4-CHF₂ |
| A-621 | 2 | 2-CHF₂, 4-CF₃ |
| A-622 | 2 | 2-CHF₂, 4-OCH₃ |
| A-623 | 2 | 2-CHF₂, 4-OCHF₂ |
| A-624 | 2 | 2-CHF₂, 4-OCF₃ |
| A-625 | 2 | 2-CHF₂, 4-CH₂OCH₃ |
| A-626 | 2 | 2-CHF₂, 4-C₂H₅ |
| A-627 | 2 | 2-CHF₂, 4-CH₂CF₃ |
| A-628 | 2 | 2-CHF₂, 4-CH═CH₂ |
| A-629 | 2 | 2-CHF₂, 4-C≡CH |
| A-630 | 2 | 2-CHF₂, 4-C≡CCH₃ |
| A-631 | 2 | 2-CHF₂, 4-C₃H₅ |
| A-632 | 2 | 2-CHF₂, 4-C(═NOCH₃)CH₃ |
| A-633 | 2 | 2-CHF₂, 4-CN |
| A-634 | 2 | 2-CHF₂, 6-F |
| A-635 | 2 | 2-CHF₂, 6-Cl |
| A-636 | 2 | 2-CHF₂, 6-Br |
| A-637 | 2 | 2-CHF₂, 6-CH₃ |
| A-638 | 2 | 2-CHF₂, 6-CHF₂ |
| A-639 | 2 | 2-CHF₂, 6-CF₃ |
| A-640 | 2 | 2-CHF₂, 6-OCH₃ |
| A-641 | 2 | 2-CHF₂, 6-OCHF₂ |
| A-642 | 2 | 2-CHF₂, 6-OCF₃ |
| A-643 | 2 | 2-CHF₂, 6-CH₂OCH₃ |
| A-644 | 2 | 2-CHF₂, 6-C₂H₅ |
| A-645 | 2 | 2-CHF₂, 6-CH₂CF₃ |
| A-646 | 2 | 2-CHF₂, 6-CH═CH₂ |
| A-647 | 2 | 2-CHF₂, 6-C≡CH |
| A-648 | 2 | 2-CHF₂, 6-C≡CCH₃ |
| A-649 | 2 | 2-CHF₂, 6-C₃H₅ |
| A-650 | 2 | 2-CHF₂, 6-C(═NOCH₃)CH₃ |
| A-651 | 2 | 2-CF₃, 3-F |
| A-652 | 2 | 2-CF₃, 3-Cl |
| A-653 | 2 | 2-CF₃, 3-Br |
| A-654 | 2 | 2-CF₃, 3-CH₃ |
| A-655 | 2 | 2-CF₃, 3-CHF₂ |
| A-656 | 2 | 2-CF₃, 3-CF₃ |
| A-657 | 2 | 2-CF₃, 3-OCH₃ |
| A-658 | 2 | 2-CF₃, 3-OCHF₂ |
| A-659 | 2 | 2-CF₃, 3-OCF₃ |
| A-660 | 2 | 2-CF₃, 3-CH₂OCH₃ |
| A-661 | 2 | 2-CF₃, 3-C₂H₅ |
| A-662 | 2 | 2-CF₃, 3-CH₂CF₃ |
| A-663 | 2 | 2-CF₃, 3-CH═CH₂ |
| A-664 | 2 | 2-CF₃, 3-C≡CH |
| A-665 | 2 | 2-CF₃, 3-C≡CCH₃ |
| A-666 | 2 | 2-CF₃, 3-C₃H₅ |
| A-667 | 2 | 2-CF₃, 3-C(═NOCH₃)CH₃ |
| A-668 | 2 | 2-CF₃, 3-CN |
| A-669 | 2 | 2-CF₃, 4-F |
| A-670 | 2 | 2-CF₃, 4-Cl |
| A-671 | 2 | 2-CF₃, 4-Br |
| A-672 | 2 | 2-CF₃, 4-CH₃ |
| A-673 | 2 | 2-CF₃, 4-CHF₂ |
| A-674 | 2 | 2-CF₃, 4-CF₃ |
| A-675 | 2 | 2-CF₃, 4-OCH₃ |
| A-676 | 2 | 2-CF₃, 4-OCHF₂ |
| A-677 | 2 | 2-CF₃, 4-OCF₃ |
| A-678 | 2 | 2-CF₃, 4-CH₂OCH₃ |
| A-679 | 2 | 2-CF₃, 4-C₂H₅ |
| A-680 | 2 | 2-CF₃, 4-CH₂CF₃ |
| A-681 | 2 | 2-CF₃, 4-CH═CH₂ |
| A-682 | 2 | 2-CF₃, 4-C≡CH |
| A-683 | 2 | 2-CF₃, 4-C≡CCH₃ |
| A-684 | 2 | 2-CF₃, 4-C₃H₅ |
| A-685 | 2 | 2-CF₃, 4-C(═NOCH₃)CH₃ |
| A-686 | 2 | 2-CF₃, 4-CN |
| A-687 | 2 | 2-CF₃, 6-F |
| A-688 | 2 | 2-CF₃, 6-Cl |
| A-689 | 2 | 2-CF₃, 6-Br |
| A-690 | 2 | 2-CF₃, 6-CH₃ |
| A-691 | 2 | 2-CF₃, 6-CHF₂ |
| A-692 | 2 | 2-CF₃, 6-CF₃ |
| A-693 | 2 | 2-CF₃, 6-OCH₃ |
| A-694 | 2 | 2-CF₃, 6-OCHF₂ |
| A-695 | 2 | 2-CF₃, 6-OCF₃ |
| A-696 | 2 | 2-CF₃, 6-CH₂OCH₃ |
| A-697 | 2 | 2-CF₃, 6-C₂H₅ |
| A-698 | 2 | 2-CF₃, 6-CH₂CF₃ |
| A-699 | 2 | 2-CF₃, 6-CH═CH₂ |
| A-700 | 2 | 2-CF₃, 6-C≡CH |
| A-701 | 2 | 2-CF₃, 6-C≡CCH₃ |
| A-702 | 2 | 2-CF₃, 6-C₃H₅ |
| A-703 | 2 | 2-CF₃, 6-C(═NOCH₃)CH₃ |
| A-704 | 2 | 2-OCH₃, 3-F |
| A-705 | 2 | 2-OCH₃, 3-Cl |
| A-706 | 2 | 2-OCH₃, 3-Br |
| A-707 | 2 | 2-OCH₃, 3-CH₃ |
| A-708 | 2 | 2-OCH₃, 3-CHF₂ |
| A-709 | 2 | 2-OCH₃, 3-CF₃ |
| A-710 | 2 | 2-OCH₃, 3-OCH₃ |
| A-711 | 2 | 2-OCH₃, 3-OCHF₂ |
| A-712 | 2 | 2-OCH₃, 3-OCF₃ |
| A-713 | 2 | 2-OCH₃, 3-CH₂OCH₃ |
| A-714 | 2 | 2-OCH₃, 3-C₂H₅ |
| A-715 | 2 | 2-OCH₃, 3-CH₂CF₃ |
| A-716 | 2 | 2-OCH₃, 3-CH═CH₂ |
| A-717 | 2 | 2-OCH₃, 3-C≡CH |
| A-718 | 2 | 2-OCH₃, 3-C≡CCH₃ |
| A-719 | 2 | 2-OCH₃, 3-C₃H₅ |
| A-720 | 2 | 2-OCH₃, 3-C(═NOCH₃)CH₃ |
| A-721 | 2 | 2-OCH₃, 3-CN |
| A-722 | 2 | 2-OCH₃, 4-F |
| A-723 | 2 | 2-OCH₃, 4-Cl |
| A-724 | 2 | 2-OCH₃, 4-Br |
| A-725 | 2 | 2-OCH₃, 4-CH₃ |
| A-726 | 2 | 2-OCH₃, 4-CHF₂ |
| A-727 | 2 | 2-OCH₃, 4-CF₃ |
| A-728 | 2 | 2-OCH₃, 4-OCH₃ |
| A-729 | 2 | 2-OCH₃, 4-OCHF₂ |
| A-730 | 2 | 2-OCH₃, 4-OCF₃ |
| A-731 | 2 | 2-OCH₃, 4-CH₂OCH₃ |
| A-732 | 2 | 2-OCH₃, 4-C₂H₅ |
| A-733 | 2 | 2-OCH₃, 4-CH₂CF₃ |
| A-734 | 2 | 2-OCH₃, 4-CH═CH₂ |
| A-735 | 2 | 2-OCH₃, 4-C≡CH |
| A-736 | 2 | 2-OCH₃, 4-C=≡CCH₃ |
| A-737 | 2 | 2-OCH₃, 4-C₃H₅ |
| A-738 | 2 | 2-OCH₃, 4-C(═NOCH₃)CH₃ |
| A-739 | 2 | 2-OCH₃, 4-CN |
| A-740 | 2 | 2-OCH₃, 6-F |
| A-741 | 2 | 2-OCH₃, 6-Cl |
| A-742 | 2 | 2-OCH₃, 6-Br |
| A-743 | 2 | 2-OCH₃, 6-CH₃ |
| A-744 | 2 | 2-OCH₃, 6-CHF₂ |
| A-745 | 2 | 2-OCH₃, 6-CF₃ |
| A-746 | 2 | 2-OCH₃, 6-OCH₃ |
| A-747 | 2 | 2-OCH₃, 6-OCHF₂ |
| A-748 | 2 | 2-OCH₃, 6-OCF₃ |
| A-749 | 2 | 2-OCH₃, 6-CH₂OCH₃ |
| A-750 | 2 | 2-OCH₃, 6-C₂H₅ |
| A-751 | 2 | 2-OCH₃, 6-CH₂CF₃ |
| A-752 | 2 | 2-OCH₃, 6-CH═CH₂ |
| A-753 | 2 | 2-OCH₃, 6-C≡CH |
| A-754 | 2 | 2-OCH₃, 6-C≡CCH₃ |
| A-755 | 2 | 2-OCH₃, 6-C₃H₅ |
| A-756 | 2 | 2-OCH₃, 6-C(═NOCH₃)CH₃ |
| A-757 | 2 | 2-CN, 3-F |
| A-758 | 2 | 2-CN, 3-Cl |
| A-759 | 2 | 2-CN, 3-Br |
| A-760 | 2 | 2-CN, 3-CH₃ |
| A-761 | 2 | 2-CN, 3-CHF₂ |
| A-762 | 2 | 2-CN, 3-CF₃ |
| A-763 | 2 | 2-CN, 3-OCH₃ |
| A-764 | 2 | 2-CN, 3-OCHF₂ |
| A-765 | 2 | 2-CN, 3-OCF₃ |
| A-766 | 2 | 2-CN, 3-CH₂OCH₃ |
| A-767 | 2 | 2-CN, 3-C₂H₅ |
| A-768 | 2 | 2-CN, 3-CH₂CF₃ |
| A-769 | 2 | 2-CN, 3-CH═CH₂ |
| A-770 | 2 | 2-CN, 3-C≡CH |
| A-771 | 2 | 2-CN, 3-C≡CCH₃ |
| A-772 | 2 | 2-CN, 3-C₃H₅ |
| A-773 | 2 | 2-CN, 3-C(═NOCH₃)CH₃ |
| A-774 | 2 | 2-CN, 3-CN |
| A-775 | 2 | 2-CN, 4-F |
| A-776 | 2 | 2-CN, 4-Cl |
| A-777 | 2 | 2-CN, 4-Br |
| A-778 | 2 | 2-CN, 4-CH₃ |
| A-779 | 2 | 2-CN, 4-CHF₂ |
| A-780 | 2 | 2-CN, 4-CF₃ |
| A-781 | 2 | 2-CN, 4-OCH₃ |
| A-782 | 2 | 2-CN, 4-OCHF₂ |
| A-783 | 2 | 2-CN, 4-OCF₃ |
| A-784 | 2 | 2-CN, 4-CH₂OCH₃ |
| A-785 | 2 | 2-CN, 4-C₂H₅ |
| A-786 | 2 | 2-CN, 4-CH₂CF₃ |
| A-787 | 2 | 2-CN, 4-CH═CH₂ |
| A-788 | 2 | 2-CN, 4-C≡CH |
| A-789 | 2 | 2-CN, 4-C≡CCH₃ |
| A-790 | 2 | 2-CN, 4-C₃H₅ |
| A-791 | 2 | 2-CN, 4-C(═NOCH₃)CH₃ |
| A-792 | 2 | 2-CN, 4-CN |
| A-793 | 2 | 2-CN, 6-F |
| A-794 | 2 | 2-CN, 6-Cl |
| A-795 | 2 | 2-CN, 6-Br |
| A-796 | 2 | 2-CN, 6-CH₃ |
| A-797 | 2 | 2-CN, 6-CHF₂ |
| A-798 | 2 | 2-CN, 6-CF₃ |
| A-799 | 2 | 2-CN, 6-OCH₃ |
| A-800 | 2 | 2-CN, 6-OCHF₂ |
| A-801 | 2 | 2-CN, 6-OCF₃ |
| A-802 | 2 | 2-CN, 6-CH₂OCH₃ |
| A-803 | 2 | 2-CN, 6-C₂H₅ |
| A-804 | 2 | 2-CN, 6-CH₂CF₃ |
| A-805 | 2 | 2-CN, 6-CH═CH₂ |
| A-806 | 2 | 2-CN, 6-C≡CH |
| A-807 | 2 | 2-CN, 6-C≡CCH₃ |
| A-808 | 2 | 2-CN, 6-C₃H₅ |
| A-809 | 2 | 2-CN, 6-C(═NOCH₃)CH₃ |

### Synthesis

The compounds can be obtained by various routes in analogy to prior art processes known (e.g EP 463488) and, advantageously, by the synthesis shown in the following schemes 1 to 4 and in the experimental part of this application.

A suitable method to prepare compounds I is illustrated in Scheme 1.

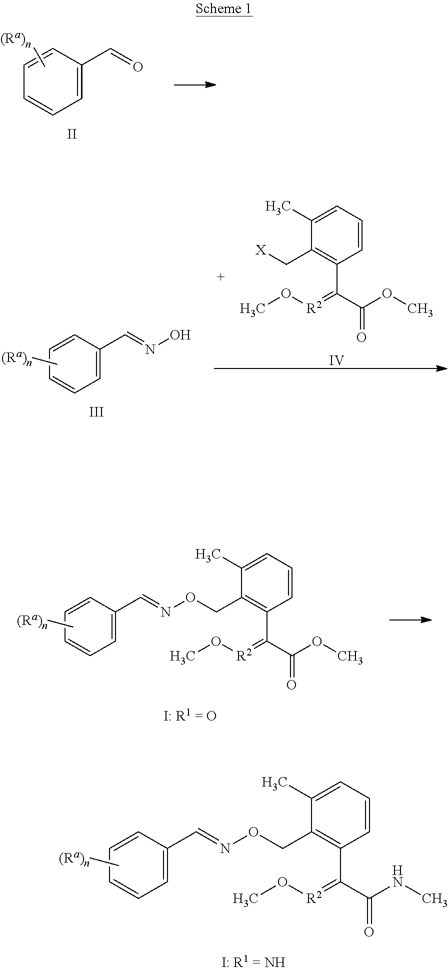

It starts with the conversion of an aldehyde II to the corresponding oxime III using hydxroxylamine hydrochloride and a base such as pyridine, sodium hydroxide or sodium acetate in polar solvents such as methanol, methanol-water mixture, or ethanol at reaction temperatures of about 60 to 100° C., preferably at about 65° C. In cases where an E/Z mixture was obtained, the isomers could be separated by purification techniques known in art (e.g. column chromatography, crystallization, distillation etc.). Then, coupling of a compound III with an intermediate IV, wherein X is a leaving group such as halogen, toluene- and methanesulfonates, preferably Cl or Br, is carried out under basic conditions using e.g. sodium hydride, cesium carbonate or potassium carbonate and using an organic solvent such as dimethyl formamide (DMF) or acetonitrile, preferably cesium carbonate as base and acetonitrile as solvent at room temperature (RT) of about 24° C. The ester compound I wherein R¹ is O can be converted to an amide of formula I wherein R¹ is NH by reaction with methyl amine (preferably 40% aq. solution) using tetrahydrofuran (THF) as solvent at RT.

Another general method to prepare the compounds I is depicted in Scheme 2.

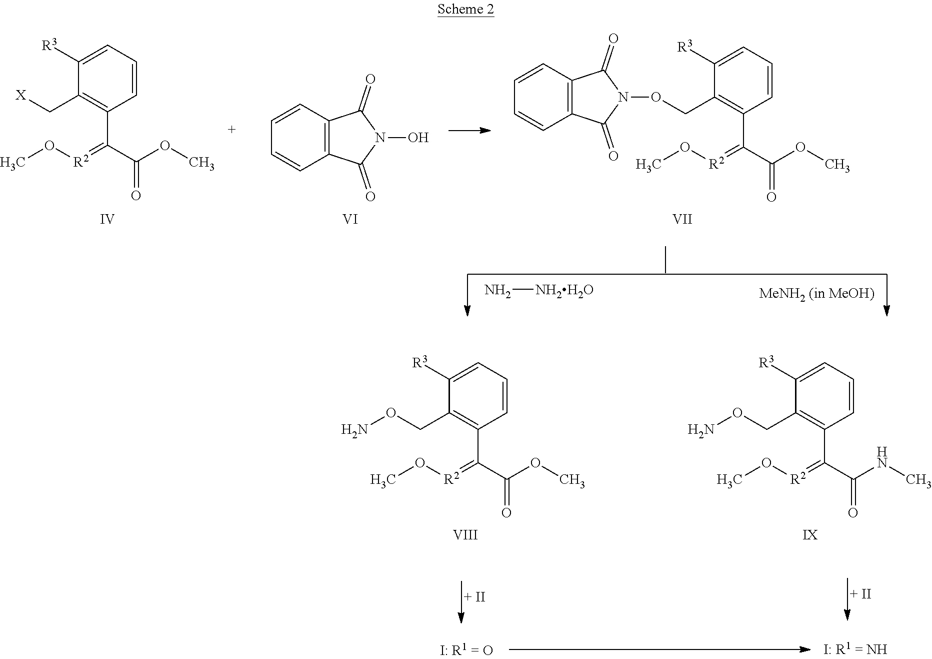

Intermediate IV is reacted with N-hydroxysuccimide VI, using a base such as triethylamine in DMF. The reaction temperature is usually 50 to 70° C. preferably about 70° C. Conversion to the corresponding O-benzylhydroxyl amine, intermediate VIII, was achieved through removal of the phthalimide group, preferably using hydrazine hydrate in methanol as solvent at about 25° C. Alternatively, removal of the phthalimide group using methyl amine in methanol as solvent at about 25° C. can provide intermediate IX. Intermediate VIII and intermediate IX, respectively can be condensed with aldehyde II using acetic acid or pyridine in methanol as solvent at temperature of about 50 to 65° C. Alternatively, the condensation could also be carried out with titanium (IV) ethoxide (Ti(OEt)₄) using THF as solvent at about 70° C. The desired product is usually accompanied by an undesired isomer, which can be removed e.g by column chromatography, crystallization.

A further method for preparation of intermediate IV is shown in Scheme 3.

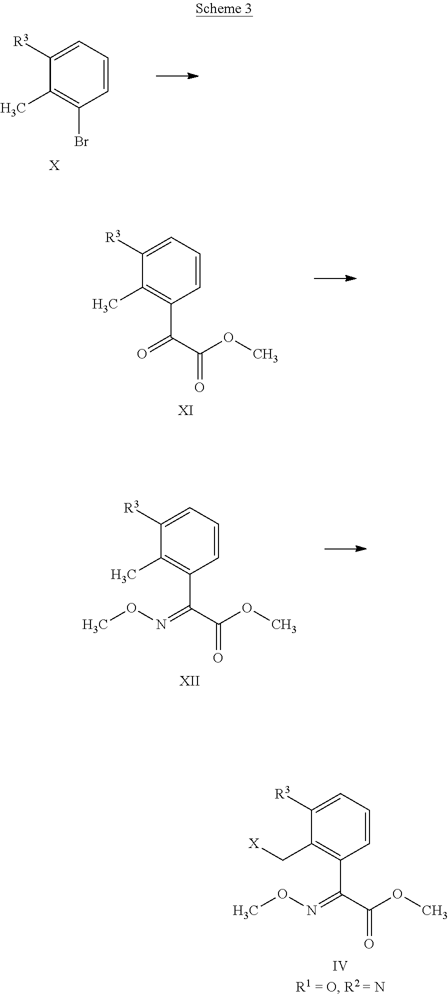

Compound XI could be obtained from X by lithium-halogen exchange or by generating Grignard reagent and further reaction with dimethyl oxalate or chloromethyl oxalate in presence of a solvent. The preferred solvent is THF, 2-methyl-THF and the temperature can be at about −70 to —78° C. Conversion of intermediate XI to intermediate XII can be achieved using N-methyl-hydroxylamine hydrochloride and a base such as pyridine or sodium acetate in polar solvents such as methanol. The reaction temperature is preferably about 65° C. An E/Z mixture is usually obtained. The isomers can be separated by purification techniques known in art (e.g. column chromatography, crystallization). Bromination of intermediate XII provides the desired intermediate compounds IV, wherein R¹ is O and R²═N. This reaction of intermediate XII with N-bromosuccinimide in solvents such as carbon tetrachloride, chlorobenzene, acetonitrile, using radical initiators such as 1,1′-azobis (cyclohexanecarbonitrile) or azobisisobutyronitrile and is carried out at temperatures of 70 to 100° C. The preferred radical initiator is 1,1′-azobis (cyclohexanecarbonitrile), preferred solvent chlorobenzene and preferred temperature 80° C.

The synthesis of compounds containing different substituents R³ follows similar sequence as in Scheme 3, wherein R³ is bromo. Coupling of intermediate III with intermediate IV, wherein R³ is bromo, provides compounds I as described above. Using standard chemical reactions, such as Suzuki or Stille reaction, the bromo group can be converted e.g. to other R³ substituents such as cycloalkyl, alkoxy and alkenyl. Additional transformations e.g. of ethenyl provide compounds I with other R³ substituents such as ethyl, CN and haloalkyl.

Most of the aldehydes of general formula II are commercially available, however for the ones which were not commercially available, preparation of these can be carried out using methods known in prior art. Scheme 4 depicts various methods known in literature for the synthesis of these aldehydes.

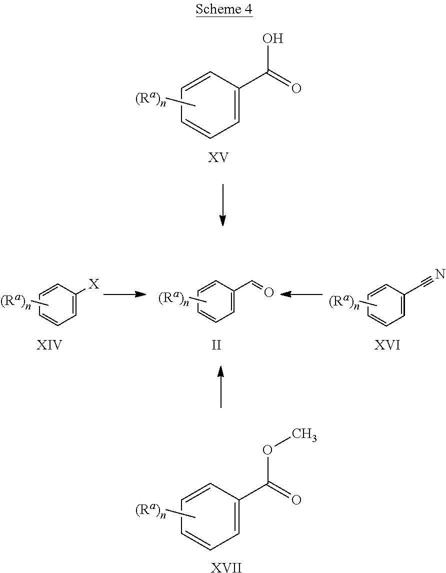

The aldehyde II can be obtained from the corresponding halogen bearing precursors XIV, wherein X is preferably bromine or iodine. Lithium-halogen exchange (J. Org. Chem. 1998, 63 (21), 7399) in compound XIII using n-butyllithium or synthesis of the corresponding Grignard reagent (Nature Comm. 2017, 8(1), 1) using THF as solvent, and subsequent reaction with N,N-dimethylformamide at about −70 to −78° C. can provide the aldehyde II (ChemCatChem. 2014, 6(9), 2692). Another method uses nitrile compounds XVI, which upon partial reduction affords aldehyde II (Synlett 1996, (2), 165; Bull. Korean Chem. Soc. 2010, 31(2), 473). Aldehyde II can also be obtained from the reduction of the corresponding carboxylic acid XV (J. Am. Chem. Soc. 1999, 121(41), 952), or the corresponding ester XVII (Tetrahedron, 2001, 57(14), 2701).

The compounds I and the compositions thereof, respectively, are suitable as fungicides effective against a broad spectrum of phytopathogenic fungi, including soil-borne fungi, in particular from the classes of Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). They can be used in crop protection as foliar fungicides, fungicides for seed dressing, and soil fungicides.

The compounds I and the compositions thereof are preferably useful in the control of phytopathogenic fungi on various cultivated plants, such as cereals, e. g. wheat, rye, barley, triticale, oats, or rice; beet, e. g. sugar beet or fodder beet; fruits, e. g. pomes (apples, pears, etc.), stone fruits (e.g. plums, peaches, almonds, cherries), or soft fruits, also called berries (strawberries, raspberries, blackberries, gooseberries, etc.); leguminous plants, e. g. lentils, peas, alfalfa, or soybeans; oil plants, e. g. oilseed rape, mustard, olives, sunflowers, coconut, cocoa beans, castor oil plants, oil palms, ground nuts, or soybeans; cucurbits, e. g. squashes, cucumber, or melons; fiber plants, e. g. cotton, flax, hemp, or jute; citrus fruits, e. g. oranges, lemons, grapefruits, or mandarins; vegetables, e. g. spinach, lettuce, asparagus, cabbages, carrots, onions, tomatoes, potatoes, cucurbits, or paprika; lauraceous plants, e. g. avocados, cinnamon, or camphor; energy and raw material plants, e. g. corn, soybean, oilseed rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e. g. flowers, shrubs, broad-leaved trees, or evergreens (conifers, eucalypts, etc.); on the plant propagation material, such as seeds; and on the crop material of these plants.

More preferably, compounds I and compositions thereof, respectively are used for controlling fungi on field crops, such as potatoes, sugar beets, tobacco, wheat, rye, barley, oats, rice, corn, cotton, soybeans, oilseed rape, legumes, sunflowers, coffee or sugar cane; fruits; vines; ornamentals; or vegetables, such as cucumbers, tomatoes, beans or squashes.

The term “cultivated plants” is to be understood as including plants which have been modified by mutagenesis or genetic engineering to provide a new trait to a plant or to modify an already present trait. Mutagenesis includes random mutagenesis using X-rays or mutagenic chemicals, but also targeted mutagenesis to create mutations at a specific locus of a plant genome. Targeted mutagenesis frequently uses oligonucleotides or proteins like CRISPR/Cas, zinc-finger nucleases, TALENs or meganucleases. Genetic engineering usually uses recombinant DNA techniques to create modifications in a genome which under natural circumstances cannot readily be obtained by cross breeding, mutagenesis or natural recombination. Typically, one or more genes are integrated into the genome of a plant to add a trait or improve or modify a trait. These integrated genes are also referred to as transgenes, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

The compounds I and compositions thereof, respectively, are particularly suitable for controlling the following causal agents of plant diseases: rusts on soybean and cereals (e.g. Phakopsora pachyrhizi and P. meibomiae on soy; Puccinia tritici, P. graminis, P. recondita and P. striiformis on wheat); molds on specialty crops, soybean, oil seed rape and sunflowers (e.g. Botrytis cinerea on strawberries and vines, Sclerotinia sclerotiorum, S. minor and S. rolfsii on oil seed rape, sunflowers and soybean); Fusarium diseases on cereals (e.g. Fusarium culmorum and F. graminearum on wheat); downy mildews on specialty crops (e.g. Plasmopara viticola on vines, Phytophthora infestans on potatoes); powdery mildews on specialty crops and cereals (e.g. Uncinula necator on vines, Erysiphe spp. on various specialty crops, Blumeria graminis on cereals); and leaf spots on cereals, soybean and corn (e.g. Zymoseptoria tritici and Septoria nodorum on cereals, S. glycines on soybean, Cercospora spp. on corn and soybean).

A further embodiment relates to the use of compound of formula (I) for combating soybean rust on soybean plants and on the plant propagation material, such as seeds, and the crop material of these plants. Soybean rust is cause by two fungal pathogens called Phakopsora pachyrhizi and P. meibomiae.

Consequently, a further embodiment relates to the use of compounds I for combating Phakopsora pachyrhizi and/or P. meibomiae on soybean plants and on the plant propagation material, such as seeds, and the crop material of these plants. A more preferred embodiment the use of compounds I for combating Phakopsora pachyrhizi on soybean plants and on the plant propagation material, such as seeds, and the crop material of these plants.

Accordingly, the present invention relates to the method for combating soybean rust (Phakopsora pachyrhizi and/or P. meibomiae), comprising:

treating the soybean plants or soybean plant propagation material to be protected against attack by Phakopsora pachyrhizi and/or P. meibomiae with an effective amount of at least one compound I, or a composition comprising such compound I.

Treatment against soybean rust can be preventive or curative.

Preferably treatment of soybean plants against soybean rust shall be preventive. Preventive treatment shall be performed when the soybean plants are at risk of infection latest shortly after the first symptoms are visible. According to one embodiment, the first treating of the soybean plants shall take place at the vegetative growth stages V3 to V4 (meaning 4 to 4 fully expanded trifoliate leaves) onwards to the reproductive growth stage R² (full bloom), more preferably place at the vegetative growth stages V6 to V8 (meaning 6 to 8 fully expanded trifoliate leaves) onwards to the reproductive growth stage R³ (beginning bloom). Depending on the disease pressure, two to four and under extreme conditions up to five applications may be necessary at application intervals of 14 to 28 days.

When employed as foliar spray against soybean rust, the amounts of the compounds I applied are, depending on the specific compound used and on the disease pressure, from 5 g to 500 g per ha, preferably from 10 to 200 per ha, more preferably from 15 to 150 g per ha, and in particular from 30 to 125 g per ha.

Furthermore, the present invention relates to the use of compounds of formula I as defined herein for combating phytopathogenic fungi containing an amino acid substitution F129L in the mitochondrial cytochrome b protein conferring resistance to Qo inhibitors.

The mutation F129L in the cytochrome b (cytb, also referred to as cob) gene shall mean any substitution of nucleotides of codon 129 encoding “F” (phenylalanine; e.g. TTT or TTC) that leads to a codon encoding “L” (leucine; e.g. TTA, TTG, TTG, CTT, CTC, CTA or CTG), for example the substitution of the first nucleotide of codon 129 ‘T’ to ‘C’ (TTT to CTT), in the cytochrome b gene resulting in a single amino acid substitution in the position 129 from F (phenylalanine) to L (leucine) (F129L) in the cytochrome b protein (Cytb). In the present invention, the mutation F129L in the cytochrome b gene shall be understood to be a single amino acid substitution in the position 129 from F (phenylalanine) to L (leucine) (F129L) in the cytochrome b protein.

Many other phytopathogenic fungi acquired the F129L mutation in the cytochrome b gene conferring resistance to Qo inhibitors, such as rusts, in particular soybean rust (Phakopsora pachyrhizi and Phakopsora meibromiae) as well as fungi from the genera Alternaria, Pyrenophora and Rhizoctonia.

Preferred fungal species are Alternaria solani, Phakopsora pachyrhizi, Phakopsora meibromiae, Pyrenophora teres, Pyrenophora tritici-repentis and Rhizoctonia solani; in particular Phakopsora pachyrhizi.

In one aspect, the present invention relates to the method of protecting plants susceptible to and/or under attack by phytopathogenic fungi containing an amino acid substitution F129L in the mitochondrial cytochrome b protein conferring resistance to Qo inhibitors, which method comprises applying to said plants, treating plant propagation material of said plants with, and/or applying to said phytopathogenic fungi, at least one compound of formula I or a composition comprising at least one compound of formula I.

According to another embodiment, the method for combating phytopathogenic fungi, comprises: a) identifying the phytopathogenic fungi containing an amino acid substitution F129L in the mitochondrial cytochrome b protein conferring resistance to Qo inhibitors, or the materials, plants, the soil or seeds that are at risk of being diseased from phytopathogenic fungi as defined herein, and b) treating said fungi or the materials, plants, the soil or plant propagation material with an effective amount of at least one compound of formula I, or a composition comprising it thereof.

The term “phytopathogenic fungi an amino acid substitution F129L in the mitochondrial cytochrome b protein conferring resistance to Qo inhibitors” is to be understood that at least 10% of the fungal isolates to be controlled contain a such F129L substitution in the mitochondrial cytochrome b protein conferring resistance to Qo inhibitors, preferably at least 30%, more preferably at least 50%, even more preferably at at least 75% of the fungi, most preferably between 90 and 100%; in particular between 95 and 100%.

The compounds I and compositions thereof, respectively, are also suitable for controlling harmful microorganisms in the protection of stored products or harvest, and in the protection of materials.

The compounds I are employed as such or in form of compositions by treating the fungi, the plants, plant propagation materials, such as seeds; soil, surfaces, materials, or rooms to be protected from fungal attack with a fungicidally effective amount of the active substances. The application can be carried out both before and after the infection of the plants, plant propagation materials, such as seeds; soil, surfaces, materials or rooms by the fungi.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, even more preferably from 0.075 to 0.75 kg per ha, and in particular from 0.1 to 0.3 kg per ha.

An agrochemical composition comprises a fungicidally effective amount of a compound I. The term “fungicidally effective amount” denotes an amount of the composition or of the compounds I, which is sufficient for controlling phytopathogenic fungi on cultivated plants or in the protection of stored products or harvest or of materials and which does not result in a substantial damage to the treated plants, the treated stored products or harvest, or to the treated materials. Such an amount can vary in a broad range and is dependent on various factors, such as the fungal species to be controlled, the treated cultivated plant, stored product, harvest or material, the climatic conditions and the specific compound I used.

The user applies the agrochemical composition usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types (see also “Catalogue of pesticide formulation types and international coding system”, Technical Monograph No. 2, 6^{th} Ed. May 2008, CropLife International) are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e.g. BR, TB, DT), granules (e. g. WG, SG, GR, FG, GG, MG), insecticidal articles (e. g. LN), as well as gel formulations for the treatment of plant propagation materials, such as seeds (e. g. GF). The compositions are prepared in a known manner, such as described by Mollet and Grubemann, Formulation technology, Wiley VCH, Weinheim, 2001; or by Knowles, New developments in crop protection product formulation, Agrow Reports DS243, T&F Informa, London, 2005.

Mixing the compounds I or the compositions comprising them in the use form as fungicides with other fungicides results in many cases in an expansion of the fungicidal spectrum of activity or in a prevention of fungicide resistance development. Furthermore, in many cases, synergistic effects are obtained (synergistic mixtures).

The following list of pesticides II, in conjunction with which the compounds I can be used, is intended to illustrate the possible combinations but does not limit them:

### A) Respiration Inhibitors

Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), metyltetraprole (A.1.25), (Z,2E)-5-[1-(2,4-dichlorophenyl)pyrazol-3-yl]-oxy-2-methoxyimino-N,3-dimethylpent-3-enamide (A.1.34), (Z,2E)-5-[1-(4-chlorophenyl)pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
inhibitors of complex III at Qᵢ site: cyazofamid (A.2.1), amisulbrom (A.2.2), [(6S,7R8R)-8-benzyl-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), florylpicoxamid (A.2.5), metarylpicoxamid (A.2.6);
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), N-[2-[2-chloro-4-(trifluoromethyl)phenoxy]phenyl]-3-(difluoromethyl)-5-fluoro-1-methyl-pyrazole-4-carboxamide (A.3.29), methyl (E)-2-[2-[(5-cyano-2-methyl-phenoxy)methyl]phenyl]-3-methoxy-prop2-enoate (A.3.30), isoflucypram (A.3.31), 2-(difluoromethyl)-N-(1,1,3-trimethyl-indan-4-yl)-pyridine-3-carboxamide (A.3.32), 2-(difluoromethyl)-N-[(3R)-1,1,3-trimethylindan-4-yl]-pyridine-3-carboxamide (A.3.33), 2-(difluoromethyl)-N-(3-ethyl-1,1-dimethyl-indan-4-yl)-pyridine-3-carboxamide (A.3.34), 2-(difluoromethyl)-N-[(3R)-3-ethyl-1,1-dimethyl-indan-4-yl]-pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]-pyridine-3-carboxamide (A.3.37), 2-(difluoromethyl)-N-(3-isobutyl-1,1-dimethyl-indan-4-yl)-pyridine-3-carboxamide (A.3.38), 2-(difluoromethyl)-N-[(3R)-3-isobutyl-1,1-dimethyl-indan-4-yl]pyridine-3-carboxamide (A.3.39) cyclobutrifluram (A.3.24);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy) phenyl]-2-pyridyl]propan-2-ol (B.1.32), fluoxytioconazole (B.1.33), ipfentrifluconazole (B.1.37), mefentrifluconazole (B.1.38), (2R)-2-[4-(4-chlorophenoxy)-2-(trifluoromethyl) phenyl]-1-(1,2,4-triazol-1-yl)propan-2-ol, (2S)-2-[4-(4-chlorophenoxy)-2-(trifluoromethyl) phenyl]-1-(1,2,4-triazol-1-yl)propan-2-ol, 2-(chloromethyl)-2-methyl-5-(p-tolylmethyl)-1-(1,2,4-triazol-1-ylmethyl) cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl) isoxazol-4-yl]-(3-pyridyl)methanol (B.1.52), 4-[[6-[2-(2,4-difluorophenyl)-1,1-difluoro-2-hydroxy-3-(1,2,4-triazol-1-yl)propyl]-3-pyridyl]oxy]benzonitrile (B.1.53), 2-[6-(4-bromophenoxy)-2-(trifluoromethyl)-3-pyridyl]-1-(1,2,4-triazol-1-yl)propan-2-ol (B.1.54), 2-[6-(4-chlorophenoxy)-2-(trifluoromethyl)-3-pyridyl]-1-(1,2,4-triazol-1-yl)propan-2-ol (B.1.55);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

tubulin inhibitors: benomyl (D.1.1), carbendazim (D.1.2), fuberidazole (D1.3), thiabendazole (D.1.4), thiophanate-methyl (D.1.5), pyridachlometyl (D.1.6), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methyl-sulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolypoxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl) oxy]-N-(2-fluoroethyl)-2-methylsulfanyl-acetamide (D.1.15), 4-(2-bromo-4-fluorophenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7), phenamacril (D.2.8);

### E) Inhibitors of Amino Acid and Protein Synthesis

methionine synthesis inhibitors: cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6);

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7), zinc thiazole (G.2.8);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
compounds affecting cell membrane permeability and fatty acides: propamocarb (G.4.1);
inhibitors of oxysterol binding protein: oxathiapiprolin (G.5.1), fluoxapiprolin (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl) pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralinl-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl) pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

inhibitors of glucan synthesis: validamycin (I.1.1), polyoxin B (I.1.2);
melanin synthesis inhibitors: pyroquilon (I.2.1), tricyclazole (I.2.2), carpropamid (I.2.3), dicyclomet (I.2.4), fenoxanil (I.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl)thiadiazole-5-carboxamide (J.1.10);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flumetylsulforim (K.1.60), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), seboctylamine (K.1.61), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3-yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), benziothiazolinone (K.1.48), bromothalonil (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), aminopyrifen (K.1.54), fluopimomide (K.1.55), N′-[5-bromo-2-methyl-6-(1-methyl-2-propoxy-ethoxy)-3-pyridyl]-N-ethyl-N-methyl-formamidine (K.1.56), N′-[4-(4,5-dichlorothiazol-2-yl)oxy-2,5-dimethyl-phenyl]-N-ethyl-N-methyl-formamidine (K.1.57), flufenoxadiazam (K.1.58), N-methyl-4-[5-(trifluoromethyl)-1,2,4-oxadiazol-3-yl]benzenecarbothioamide (K.1.59), N-methoxy-N-[[4-[5-(trifluoromethyl)-1,2,4-oxadiazol-3-yl]phenyl]methyl]cyclopropanecarboxamide (K.1.60; WO2018/177894, WO 2020/212513), N-((4-[5-(trifluoromethyl)-1,2,4-oxadiazol-3-yl]phenyl) methyl)propanamide (K.1.62), 3,3,3-trifluoro-N-[[3-fluoro-4-[5-(trifluoromethyl)-1,2,4-oxadiazol-3-yl]phenyl]methyl]propanamide (K.1.63), 3,3,3-trifluoro-N-[[2-fluoro-4-[5-(trifluoromethyl)-1,2,4-oxadiazol-3-yl]phenyl]methyl]propanamide (K.1.64), N-[2,3-difluoro-4-[5-(trifluoromethyl)-1,2,4-oxadiazol-3-yl]benzyl]butanamide (K.1.65), N-[[2,3-difluoro-4-[5-(trifluoromethyl)-1,2,4-oxadiazol-3-yl]phenyl]methyl]-3,3,3-trifluoro-propanamide (K.1.66), 1-methoxy-1-methyl-3-[[4-[5-(trifluoromethyl)-1,2,4-oxadiazol-3-yl]phenyl]methyl]-urea (K.1.67), 1,1-diethyl-3-[[4-[5-[trifluoromethyl]-1,2,4-oxadiazol-3-yl]phenyl]methyl]urea (K.1.68), N,2-dimethoxy-N-[[4-[5-(trifluoromethyl)-1,2,4-oxadiazol-3-yl]phenyl]methyl]propanamide (K.1.69), N-ethyl-2-methyl-N-[[4-[5-(trifluoromethyl)-1,2,4-oxadiazol-3-yl]phenyl]methyl]propanamide (K.1.70), 1-methoxy-3-methyl-1-[[4-[5-(trifluoromethyl)-1,2,4-oxadiazol-3-yl]-phenyl]methyl]urea (K.1.71), 1-[[4-[5-(trifluoromethyl)-1,2,4-oxadiazol-3-yl]phenyl]methyl]pyrrolidin-2-one (K.1.72), 1-[[4-[5-(trifluoromethyl)-1,2,4-oxadiazol-3-yl]phenyl]methyl]piperidin-2-one (K.1.73), 4-[[4-[5-(trifluoromethyl)-1,2,4-oxadiazol-3-yl]phenyl]methyl]morpholin-3-one (K.1.74), 4,4-dimethyl-2-[[4-[5-(trifluoromethyl)-1,2,4-oxadiazol-3-yl]phenyl]methyl]isoxazolidin-3-one (K.1.75), 2-[[4-[5-(trifluoromethyl)-1,2,4-oxadiazol-3-yl]phenyl]methyl]isoxazolidin-3-one (K.1.76), 5,5-dimethyl-2-[[4-[5-(trifluoromethyl)-1,2,4-oxadiazol-3-yl]phenyl]methyl]-isoxazolidin-3-one (K.1.77), 3,3-dimethyl-1-[[4-[5-(trifluoromethyl)-1,2,4-oxadiazol-3-yl]phenyl]methyl]piperidin-2-one (K.1.78), 2-[[4-[5-(trifluoromethyl)-1,2,4-oxadiazol-3-yl]phenyl]methyl]oxazinan-3-one (K.1.79), 1-[[3-fluoro-4-[5-(trifluoromethyl)-1,2,4-oxadiazol-3-yl]phenyl]-methyl]azepan-2-one (K.1.80), 4,4-dimethyl-1-[[4-[5-(trifluoromethyl)-1,2,4-oxadiazol-3-yl]-phenyl]methyl]pyrrolidin-2-one (K.1.81), 5-methyl-1-[[4-[5-(trifluoromethyl)-1,2,4-oxadiazol-3-yl]phenyl]methyl]pyrrolidin-2-one (K.1.82), ethyl 1-[[4-[5-(trifluoromethyl)-1,2,4-oxadiazol-3-yl]phenyl]methyl]pyrazole-4-carboxylate (K.1.83), N-methyl-1-[[4-[5-(trifluoromethyl)-1,2,4-oxadiazol-3-yl]phenyl]methyl]pyrazole-4-carboxamide (K.1.84), N,N-dimethyl-1-[4-[5-(trifluoromethyl)-1,2,4-oxadiazol-3-yl]benzyl]-1H-1,2,4-triazol-3-amine (K.1.85), N-methoxy-N-methyl-1-[[4-[5-(trifluoromethyl)-1,2,4-oxadiazol-3-yl]phenyl]methyl]pyrazole-4-carboxamide (K.1.86), propyl-1-[[4-[5-(trifluoromethyl)-1,2,4-oxadiazol-3-yl]phenyl]methyl]-pyrazole-4-carboxamide (K.1.87), N-methoxy-1-[[4-[5-(trifluoromethyl)-1,2,4-oxadiazol-3-yl]phenyl]methyl]pyrazole-4-carboxamide (K.1.88), N-allyl-N-[[4-[5-(trifluoromethyl)-1,2,4-oxadiazol-3-yl]phenyl]methyl]propanamide (K.1.89), 3-ethyl-1-methoxy-1-[[4-[5-(trifluoromethyl)-1,2,4-oxadiazol-3-yl]phenyl]methyl]urea (K.1.90), 1,3-dimethoxy-1-[4-[[5-(trifluoromethyl)-1,2,4-oxadiazol-3-yl]phenyl]methyl]urea (K.1.91), N-allyl-N-[[4-[5-(trifluoromethyl)-1,2,4-oxadiazol-3-yl]phenyl]methyl]acetamide (K.1.92), N-[4-[5-(trifluoromethyl)-1,2,4-oxadiazol-3-yl]benzyl]cyclopropanecarboxamide (K.1.93), 1-methyl-3-[[4-[5-(trifluoromethyl)-1,2,4-oxadiazol-3-yl]phenyl]methyl]urea (K.1.94), N′-[2-chloro-4-(2-fluorophenoxy)-5-methyl-phenyl]-N-ethyl-N-methyl-formamidine (K.1.95).

In the binary mixtures the weight ratio of the component 1) and the component 2) generally depends from the properties of the components used, usually it is in the range of from 1:10,000 to 10,000:1, often from 1:100 to 100:1, regularly from 1:50 to 50:1, preferably from 1:20 to 20:1, more preferably from 1:10 to 10:1, even more preferably from 1:4 to 4:1 and in particular from 30 1:2 to 2:1. According to further embodiments, the weight ratio of the component 1) and the component 2) usually is in the range of from 1000:1 to 1:1, often from 100:1 to 1:1, regularly from 50:1 to 1:1, preferably from 20:1 to 1:1, more preferably from 10:1 to 1:1, even more preferably from 4:1 to 1:1 and in particular from 2:1 to 1:1. According to further embodiments, the weight ratio of the component 1) and the component 2) usually is in the range of from 20,000:1 to 1:10, often from 10,000:1 to 1:1, regularly from 5,000:1 to 5:1, preferably from 5,000:1 to 10:1, more preferably from 2,000:1 to 30:1, even more preferably from 2,000:1 to 100:1 and in particular from 1,000:1 to 100:1. According to further embodiments, the weight ratio of the component 1) and the component 2) usually is in the range of from 1:1 to 1:1000, often from 1:1 to 1:100, regularly from 1:1 to 1:50, preferably from 1:1 to 1:20, more preferably from 1:1 to 1:10, even more preferably from 1:1 to 1:4 and in particular from 1:1 to 1:2. According to further embodiments, the weight ratio of the component 1) and the component 2) usually is in the range of from 10:1 to 1:20,000, often from 1:1 to 1:10,000, regularly from 1:5 to 1:5,000, preferably from 1:10 to 1:5,000, more preferably from 1:30 to 1:2,000, even more preferably from 1:100 to 1:2,000 to and in particular from 1:100 to 1:1,000.

In the ternary mixtures, i.e. compositions comprising the component 1) and component 2) and a compound III (component 3), the weight ratio of component 1) and component 2) depends from the properties of the active substances used, usually it is in the range of from 1:100 to 100:1, regularly from 1:50 to 50:1, preferably from 1:20 to 20:1, more preferably from 1:10 to 10:1 and in particular from 1:4 to 4:1, and the weight ratio of component 1) and component 3) usually it is in the range of from 1:100 to 100:1, regularly from 1:50 to 50:1, preferably from 1:20 to 20:1, more preferably from 1:10 to 10:1 and in particular from 1:4 to 4:1. Any further active components are, if desired, added in a ratio of from 20:1 to 1:20 to the component 1). These ratios are also suitable for mixtures applied by seed treatment.

Preference is given to mixtures comprising as component 2) at least one active substance selected from inhibitors of complex III at Qₒ site in group A), more preferably selected from compounds (A.1.1), (A.1.4), (A.1.8), (A.1.9), (A.1.10), (A.1.12), (A.1.13), (A.1.14), (A.1.17), (A.1.21), (A.1.25), (A.1.34) and (A.1.35); particularly selected from (A.1.1), (A.1.4), (A.1.8), (A.1.9), (A.1.13), (A.1.14), (A.1.17), (A.1.25), (A.1.34) and (A.1.35).

Preference is also given to mixtures comprising as component 2) at least one active substance selected from inhibitors of complex III at Q, site in group A), more preferably selected from compounds (A.2.1), (A.2.3) and (A.2.4); particularly selected from (A.2.3) and (A.2.4).

Preference is also given to mixtures comprising as component 2) at least one active substance selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.28), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.12), (A.3.15), (A.3.17), (A.3.19), (A.3.22), (A.3.23), (A.3.31), (A.3.32), (A.3.33), (A.3.34), (A.3.35), (A.3.36), (A.3.37), (A.3.38) and (A.3.39).

Preference is also given to mixtures comprising as component 2) at least one active substance selected from other respiration nhibitors in group A), more preferably selected from compounds (A.4.5) and (A.4.11); in particular (A.4.11).

Preference is also given to mixtures comprising as component 2) at least one active substance selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.34), (B.1.37), (B.1.38), (B.1.43), (B.1.46), (B.1.53), (B.1.54) and (B.1.55); in particlar from (B.1.5), (B.1.8), (B.1.10), (B.1.17), (B.1.22), (B.1.23), (B.1.25), (B.1.33), (B.1.34), (B.1.37), (B.1.38), (B.1.43) and (B.1.46).

Preference is also given to mixtures comprising as component 2) at least one active substance selected from Delta14-reductase inhibitors in group B), more preferably selected from compounds (B.2.4), (B.2.5), (B.2.6) and (B.2.8); in particular (B.2.4).

Preference is also given to mixtures comprising as component 2) at least one active substance selected from phenylamides and acyl amino acid fungicides in group C), more preferably selected from compounds (C.1.1), (C.1.2), (C.1.4) and (C.1.5); particularly selected from (C.1.1) and (C.1.4).

Preference is also given to mixtures comprising as component 2) at least one active substance selected from other nucleic acid synthesis inhibitors in group C), more preferably selected from compounds (C.2.6), (C.2.7) and (C.2.8).

Preference is also given to mixtures comprising as component 2) at least one active substance selected from group D), more preferably selected from compounds (D.1.1), (D.1.2), (D.1.5), (D.2.4) and (D.2.6); particularly selected from (D.1.2), (D.1.5) and (D.2.6).

Preference is also given to mixtures comprising as component 2) at least one active substance selected from group E), more preferably selected from compounds (E.1.1), (E.1.3), (E.2.2) and (E.2.3); in particular (E.1.3).

Preference is also given to mixtures comprising as component 2) at least one active substance selected from group F), more preferably selected from compounds (F.1.2), (F.1.4) and (F.1.5).

Preference is also given to mixtures comprising as component 2) at least one active substance selected from group G), more preferably selected from compounds (G.3.1), (G.3.3), (G.3.6), (G.5.1), (G.5.3), (G.5.4), (G.5.5), G.5.6), G.5.7), (G.5.8), (G.5.9), (G.5.10) and (G.5.11); particularly selected from (G.3.1), (G.5.1) and (G.5.3).

Preference is also given to mixtures comprising as component 2) at least one active substance selected from group H), more preferably selected from compounds (H.2.2), (H.2.3), (H.2.5), (H.2.7), (H.2.8), (H.3.2), (H.3.4), (H.3.5), (H.4.9) and (H.4.10); particularly selected from (H.2.2), (H.2.5), (H.3.2), (H.4.9) and (H.4.10).

Preference is also given to mixtures comprising as component 2) at least one active substance selected from group I), more preferably selected from compounds (1.2.2) and (1.2.5).

Preference is also given to mixtures comprising as component 2) at least one active substance selected from group J), more preferably selected from compounds (J.1.2), (J.1.5), (J.1.8), (J.1.11) and (J.1.12); in particular (J.1.5).

Preference is also given to mixtures comprising as component 2) at least one active substance selected from group K), more preferably selected from compounds (K.1.41), (K.1.42), (K.1.44), (K.1.47), (K.1.57), (K.1.58) and (K.1.59); particularly selected from (K.1.41), (K.1.44), (K.1.47), (K.1.57), (K.1.58) and (K.1.59).

The compositions comprising mixtures of active ingredients can be prepared by usual means, e. g. by the means given for the compositions of compounds I.

### EXAMPLES

### Synthetic Process

### Example 33: Methyl (2E)-2-[2-[[(E)-[4-fluoro-2-(trifluoromethyl)phenyl]methyleneamino]oxymethyl]-3-methyl-phenyl]-2-methoxyimino-acetate

### Step 1: (1E)-4-fluoro-2-(trifluoromethyl)benzaldehyde oxime

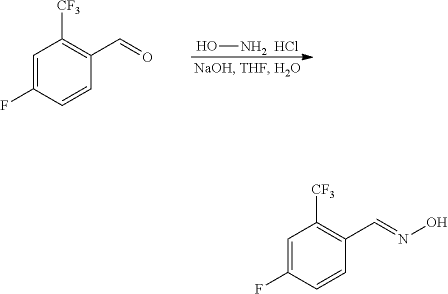

To a solution of 4-fluoro-2-(trifluoromethyl)benzaldehyde (1 g, 5.2 mmol) in MeOH/H₂O (10 mL/2 mL), hydroxylamine hydrochloride (729 mg, 10.42 mmol), NaOH aq. (4N in water, 3.25 mL, 13 mmol) were added under N₂. The mixture was stirred for 4 h at 80° C. under N₂. Thin layer chromatography (TLC) (petroleum Ether (PE):ethyl acetate (EtOAc)=5:1) showed the reaction was completed. The reaction mixture was concentrated, then dissolved in EtOAc (10 mL) and H₂O (10 mL). The aqueous phase was extracted with EtOAc (2×8 mL), washed with brine (20 mL), dried over Na₂SO₄ and concentrated to give (1E)-4-fluoro-2-(trifluoromethyl) benzaldehyde oxime (450 mg, 42.05%) as white solid.

¹H NMR (400 MHz, CHCl₃-d): δ 8.45 (d, J=2.0 Hz, 1H), 8.04 (dd, J=5.5, 8.8 Hz, 1H), 7.46 (s, 1H), 7.41 (dd, J=2.5, 8.8 Hz, 1H), 7.30 (br d, J=2.5 Hz, 1H).

### Step 2: Methyl (2E)-2-[2-[[(E)-[4-fluoro-2-(trifluoromethyl)phenyl]methyleneamino]oxymethyl]-3-methyl-phenyl]-2-methoxyimino-acetate

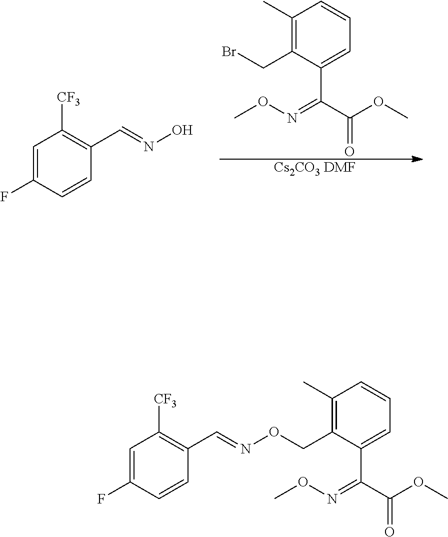

To a solution of (1E)-4-fluoro-2-(trifluoromethyl)benzaldehyde oxime (450 mg, 2.17 mmol) in DMF (6 mL), methyl (2E)-2[2-(bromomethyl)-3-methyl-phenyl]-2-methoxyimino-acetate (652 mg, 2.17 mmol) and CS₂CO₃ (1.172 g, 5.43 mmol) were added. The mixture was stirred for 5 h at about 15° C. TLC (PE:EtOAc=5:1) showed the reaction was completed. The mixture was quenched with H₂O (15 mL), extracted with EtOAc (2×20 mL), and the organic phase was washed with brine (50 mL), dried over Na₂SO₄, concentrated and purified by Prep-HPLC to give the title compound (520 mg, 56.16%) as yellow solid. ¹H NMR (400 MHz, CHCl₃-d) δ 8.30 (q, J=2.0 Hz, 1H), 8.03 (dd, J=5.5, 8.8 Hz, 1H), 7.38-7.29 (m, 3H), 7.27-7.21 (m, 1H), 7.04 (dd, J=1.3, 7.2 Hz, 1H), 5.13 (br s, 2H), 4.03-4.03 (m, 3H), 3.83-3.82 (m, 3H), 2.49 (s, 3H).

### Example 34: (2E)-2-[2-[[(E)-[4-fluoro-2-(trifluoromethyl)phenyl]methyleneamino]oxymethyl]-3-methyl-phenyl]-2-methoxyimino-N-methyl-acetamide

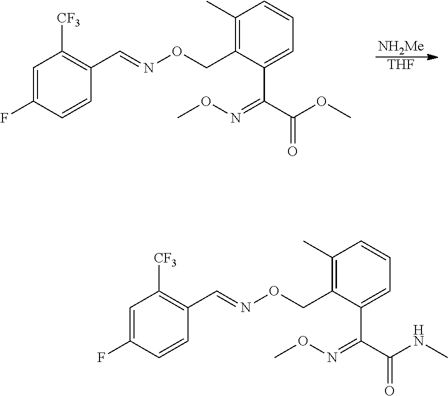 [SMILES: CO/N=C(/C(=O)OC)c1cccc(C)c1CO/N=C/c1ccc(F)cc1C(F)(F)F ; CNC(=O)/C(=N/OC)c1cccc(C)c1CO/N=C/c1ccc(F)cc1C(F)(F)F ; "NH2Me" ; "THF"]

To a solution of methyl (2E)-2-[2-[[(E)-[4-fluoro-2-(trifluoromethyl)phenyl]methyleneamino]-oxymethyl]-3-methyl-phenyl]-2-methoxyimino-acetate (310 mg, 0.728 mmol) in THF (4 mL), MeNH₂ (282 mg, 3.638 mmol) was added and the mixture was stirred for 16 h at about 15° C. TLC (PE:EtOAc=5:1) showed that the reaction was completed. The mixture was quenched with H₂O (10 mL), extracted with EtOAc (3×15 mL), the organic phase was washed with brine (50 mL), dried over Na₂SO₄ and concentrated to give the title compound (305 mg, 98.7%) as yellow solid.

¹H NMR (400 MHz, CHCl₃-d) δ 8.31 (br d, J=2.0 Hz, 1H), 8.04 (dd, J=5.6, 8.7 Hz, 1H), 7.39-7.27 (m, 3H), 7.26-7.21 (m, 1H), 7.04 (d, J=7.3 Hz, 1H), 6.75 (br d, J=4.1 Hz, 1H), 5.12 (s, 2H), 3.94 (s, 3H), 2.90 (d, J=5.0 Hz, 3H), 2.48 (s, 3H).

### Example 35: Methyl (2E)-2-[2-[[(E)-[4-bromo-2-(trifluoromethyl)phenyl]methyleneamino]oxymethyl]-3-methyl-phenyl]-2-methoxyimino-acetate

### Step 1: (1E)-4-bromo-2-(trifluoromethyl)benzaldehyde oxime

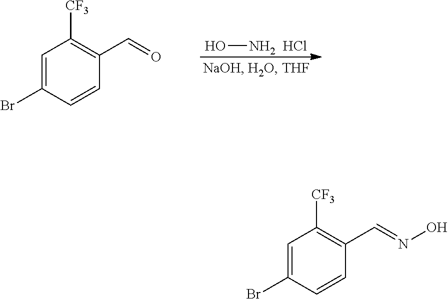

To a solution of 4-bromo-2-(trifluoromethyl)benzaldehyde (1 g, 3.968 mmol) in THF (10 mL); HONH₂·HCl (556 mg, 7.937 mmol) and NaOH aq. (4N in water, 2.5 mL, 9.92 mmol) were added. The mixture was stirred for 4 h at about 80° C. under N₂. TLC (PE:EtOAc=5:1) showed that the reaction was completed. The reaction mixture was quenched with H₂O 15 mL), extracted with EtOAc (2×10 mL). The organic phase was washed with brine (25 mL), dried over Na₂SO₄ and concentrated to give (1E)-4-bromo-2-(trifluoromethyl)benzaldehyde oxime (1 g, 94.34%) as white solid. ¹H NMR: (400 MHz, CHCl₃-d) δ 8.44 (br d, J=1.9 Hz, 1H), 7.90 (d, J=8.5 Hz, 1H), 7.84 (d, J=1.4 Hz, 1H), 7.73-7.66 (m, 2H).

### Step 2: Methyl (2E)-2-[2-[[(E)-[4-bromo-2-(trifluoromethyl)phenyl]methyleneamino]oxymethyl]-3-methyl-phenyl]-2-methoxyimino-acetate

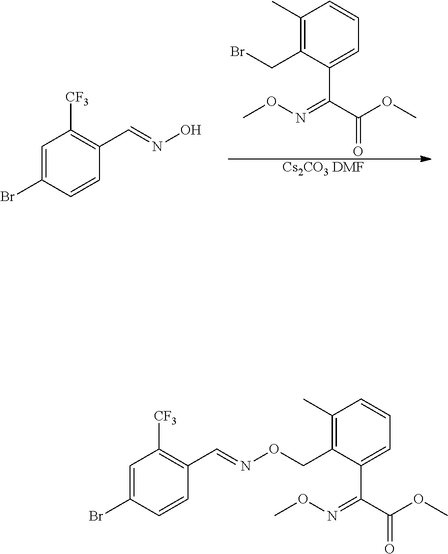

To a solution of (1E)-4-bromo-2-(trifluoromethyl)benzaldehyde oxime (1.18 g, 4.42 mmol) in DMF (15 mL), methyl (2E)-2-[2-(bromomethyl)-3-methyl-phenyl]-2-methoxyimino-acetate (1.32 g, 4.42 mmol) and Cs₂CO₃ (3.6 g, 11.08 mmol) were added. The mixture was stirred for 4 h at about 20° C. TLC (PE: EtOAc=4:1) showed that the reaction was completed. The reaction mixture was quenched with H₂O (25 mL), extracted with EtOAc (2×20 mL). The organic phase was washed with brine (15 mL), dried over Na₂SO₄, concentrated and purified by silica gel column (PE: EtOAc=100:0 to 80:20) to give the title compound (1.98 g, yield: 92.1%) as a white solid. ¹H NMR (CDCl₃ Varian_D_400 MHz): δ 8.28 (d, J=2.19 Hz, 1 H) 7.90 (d, J=8.55 Hz, 1 H) 7.79 (d, J=1.97 Hz, 1 H) 7.66 (dd, J=8.44, 2.08 Hz, 1 H) 7.31-7.36 (m, 1 H) 7.27-7.31 (m, 1 H) 7.03 (dd, J=7.34, 1.21 Hz, 1 H) 5.13 (br s, 2 H) 4.03 (s, 3 H) 3.82 (s, 3 H) 2.49 (s, 3 H).

### Example 36: (2E)-2-[2-[[(E)-[4-bromo-2-(trifluoromethyl)phenyl]methyleneamino]oxymethyl]-3-methyl-phenyl]-2-methoxyimino-N-methyl-acetamide

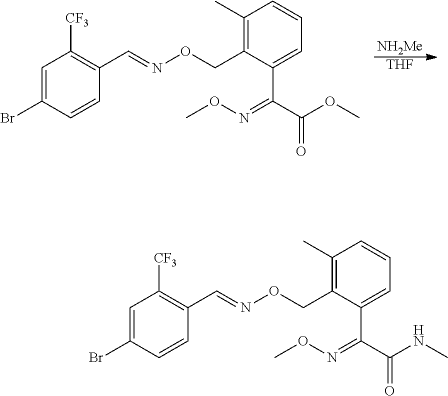 [SMILES: CO/N=C(/C(=O)OC)c1cccc(C)c1CO/N=C/c1ccc(Br)cc1C(F)(F)F ; CNC(=O)/C(=N/OC)c1cccc(C)c1CO/N=C/c1ccc(Br)cc1C(F)(F)F ; "NH2Me" ; "THF"]

To a solution of methyl (2E)-2-[2-[[(E)-[4-bromo-2-(trifluoromethyl)phenyl]methyleneamino]-oxymethyl]-3-methyl-phenyl]-2-methoxyimino-acetate (360 mg, 0.74 mmol) in THF (5 mL), MeNH₂ (239 mg, 3.2 mmol) was added. The mixture was stirred for 16 h at about 20° C. TLC (PE: EtOAc=1:1) showed that the reaction was completed. The reaction mixture was quenched with NHCl₄ aq. (15 mL) and extracted with EtOAc (15×2 mL). The organic phase was washed with brine (15 mL), dried over Na₂SO₄ and concentrated to give the title compound (566 mg, yield: 94.3%) as white solid. ¹H NMR (CDCl₃ Bruker_J_400 MHz): δ 8.29 (d, J=1.88 Hz, 1 H) 7.91 (d, J=8.50 Hz, 1 H) 7.79 (d, J=1.50 Hz, 1 H) 7.66 (br d, J=8.50 Hz, 1 H) 7.27-7.37 (m, 2 H) 7.03 (d, J=7.25 Hz, 1 H) 6.74 (br d, J=4.13 Hz, 1 H) 5.13 (s, 2 H) 3.94 (s, 3 H) 2.90 (d, J=5.00 Hz, 3 H) 2.48 (s, 3 H).

### Example 37: Methyl (2E)-2-methoxyimino-2-[3-methyl-2-[[(E)-[4-methyl-2-(trifluoromethyl)phenyl]methyleneamino]oxymethyl]phenyl]acetate

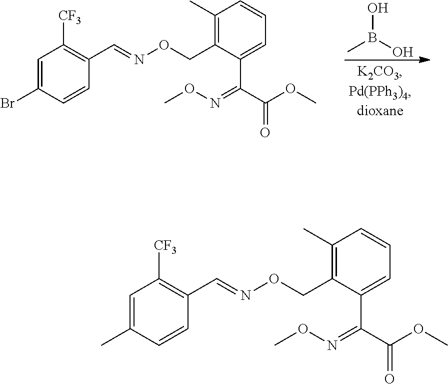

To a solution of methyl (2E)-2-[2-[[(E)-[4-bromo-2-(trifluoromethyl)phenyl]methyleneamino]oxymethyl]-3-methyl-phenyl]-2-methoxyimino-acetate (1.24 g, 2.55 mmol) in dioxane (25 mL) under Nitrogen, methyl boronic acid (740 mg, 12.75 mmol), K₂CO₃ (880 mg, 6.38 mmol) and Pd (PPh₃)₄ (143 mg, 0.13 mmol) were added. The reaction mixture was stirred for 6 hat about 100° C. TLC (PE:EtOAc=3:1) showed that the reaction was completed. The reaction mixture was quenched with H₂O (30 mL) and extracted with EtOAc (2×20 mL). The organic phase was washed with brine (20 mL), dried over Na₂SO₄, concentrated and purified by silica gel column (PE:EtOAc=100:0 to 85:15) to give the title compound (0.8 g, yield: 74.3%) as white solid. ¹H NMR (CDCl₃ Varian_D_400 MHz):δ 8.33 (d, J=2.19 Hz, 1 H) 7.91 (d, J=7.89 Hz, 1 H) 7.45 (s, 1 H) 7.27-7.36 (m, 3 H) 7.03 (dd, J=7.34, 1.43 Hz, 1 H) 5.12 (s, 2 H) 4.03 (s, 3 H) 3.82 (s, 3 H) 2.50 (s, 3 H) 2.41 (s, 3 H).

### Example 38: (2E)-2-methoxyimino-N-methyl-2-[3-methyl-2-[[(E)-[4-methyl-2-(trifluoromethyl)-phenyl]methyleneamino]oxymethyl]phenyl]acetamide

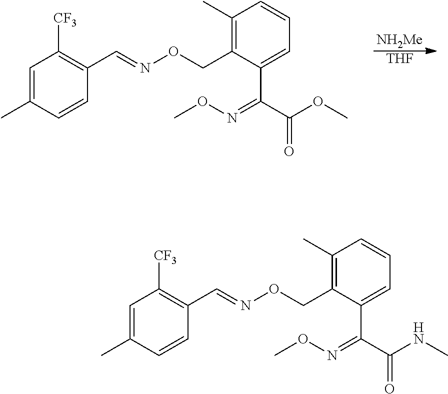

To a solution of methyl (2E)-2-methoxyimino-2-[3-methyl-2-[[(E)-[4-methyl-2-(trifluoromethyl)-phenyl]methyleneamino]oxymethyl]phenyl]acetate (400 mg, 0.95 mmol) in THF (3 mL), MeNH₂ (˜33% in water, 368 mg, 4.8 mmol) was added. The mixture was stirred for 16 h at about 20° C. TLC (PE:EtOAc=5:1) showed that the reaction was completed. The mixture was quenched with NHCl₄ aq (15 mL) and extracted with EtOAc (2×10 mL). The organic phase was washed with brine (10 mL), dried over Na₂SO₄ and concentrated to give the title compound (280 mg, yield: 70%) as yellow solid. ¹H NMR (CDCl₃ Bruker_K_400 MHz):δ 8.34 (d, J=2.13 Hz, 1 H) 7.90 (d, J=8.00 Hz, 1 H) 7.45 (s, 1 H) 7.29-7.36 (m, 2 H) 7.27 (s, 1 H) 7.00-7.08 (m, 1 H) 6.72 (br d, J=4.25 Hz, 1 H) 5.13 (s, 2 H) 3.94 (s, 3 H) 2.89 (d, J=5.00 Hz, 3 H) 2.48 (s, 3 H) 2.41 (s, 3 H).

The following examples in Table S1 and S2 were synthesized as described above and characterized by LCMS as described in Table L or by ¹H-NMR.

TABLE L
LCMS Methods

| LCMS Method A | LCMS Method B |
|---|---|
| Column: Agilent Eclipse Plus C18 | Column: Kinetex XB C18 |
| (50 mm × 4.6 mm × 3 μ) | (50 mm × 2.1 mm × 1.7 μ) |
| Mobile Phase: | Mobile Phase: |
| A: 10 mM Ammonium formate in water. | A: Water + 0.1% TFA. |
| B: 0.1% Formic acid in acetonitrile | B: Acetonitrile |
| Gradient: 10% B to 100% B in 1.5 | Gradient: 5% B to 100% B in |
| min. Hold 1 min 100% B. 1 min 10% B. | 1.5 min. |
| Run time: 3.50 or 3.75 min. | Flow: 0.8 ml/min to 1.0 |
| Flow: 1.2 ml/min; | ml/min in 1.5 min; |
| Column oven: 30° C./40° C. | Column oven: 60° C. |

Device details for LCMS Method A and B
LCMS2020 (Shimadzu), Ionization source: ESI; Mass range: 100-800 amu; Polarity: Dual (positive and negative simultaneous scan); Mode: Scan; LC System: Nexera High pressure gradient system, Binary pump; Detector: PDA; Scanning wavelength: 220 nm/max plot

TABLE S1

| | | LCMS | | |
|---|---|---|---|---|
| Compound | | Rₜ | | |
| No. | Structure | [min] | Mass | Meth. |
| 1 | 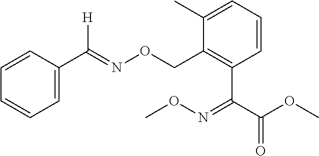 [SMILES: CO/N=C(/C(=O)OC)c1cccc(C)c1CO/N=C/c1ccccc1] | 1.254 | 341 | B |
| 2 | 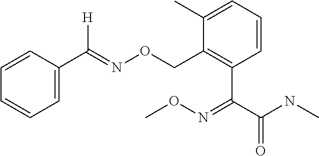 [SMILES: CNC(=O)/C(=N/OC)c1cccc(C)c1CO/N=C/c1ccccc1] | 1.158 | 340 | B |
| 3 | 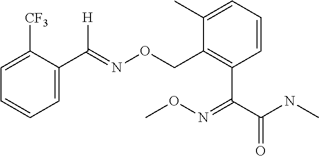 [SMILES: CNC(=O)/C(=N/OC)c1cccc(C)c1CO/N=C/c1ccccc1C(F)(F)F] | 2.08 | 408 | A |
| 4 | 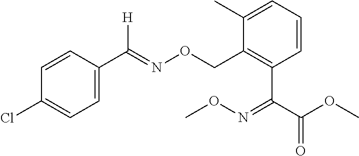 [SMILES: CO/N=C(/C(=O)OC)c1cccc(C)c1CO/N=C/c1ccc(Cl)cc1] | 2.176 | 375 | A |
| 5 | 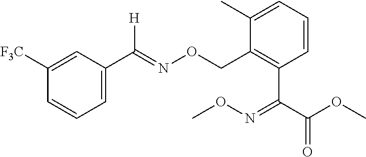 [SMILES: CO/N=C(/C(=O)OC)c1cccc(C)c1CO/N=C/c1cccc(C(F)(F)F)c1] | 2.17 | 409 | A |
| 6 | 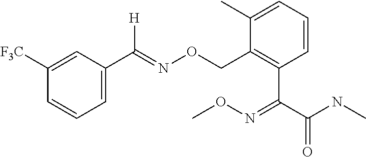 [SMILES: CNC(=O)/C(=N/OC)c1cccc(C)c1CO/N=C/c1cccc(C(F)(F)F)c1] | 2.059 | 408 | A |
| 7 | 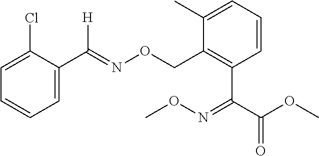 [SMILES: CO/N=C(/C(=O)OC)c1cccc(C)c1CO/N=C/c1ccccc1Cl] | 2.187 | 375 | A |
| 8 | 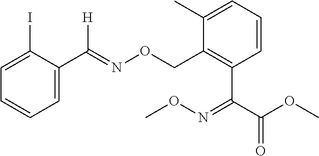 [SMILES: CO/N=C(/C(=O)OC)c1cccc(C)c1CO/N=C/c1ccccc1I] | 2.229 | 467 | A |
| 9 | 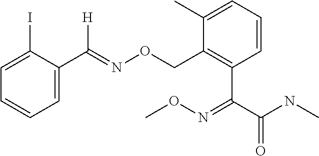 [SMILES: CNC(=O)/C(=N/OC)c1cccc(C)c1CO/N=C/c1ccccc1I] | 2.101 | 466 | A |
| 10 | 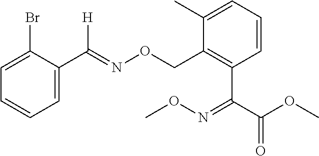 [SMILES: CO/N=C(/C(=O)OC)c1cccc(C)c1CO/N=C/c1ccccc1Br] | 2.208 | 421 | A |
| 11 | 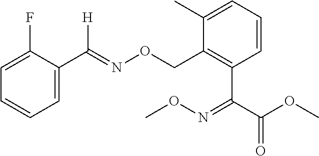 [SMILES: CO/N=C(/C(=O)OC)c1cccc(C)c1CO/N=C/c1ccccc1F] | 2.112 | 359 | A |
| 12 | 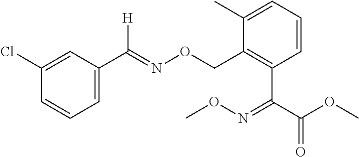 [SMILES: CO/N=C(/C(=O)OC)c1cccc(C)c1CO/N=C/c1cccc(Cl)c1] | 2.176 | 375 | A |
| 13 | 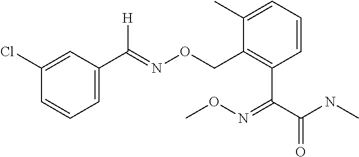 [SMILES: CNC(=O)/C(=N/OC)c1cccc(C)c1CO/N=C/c1cccc(Cl)c1] | 2.048 | 374 | A |
| 14 | 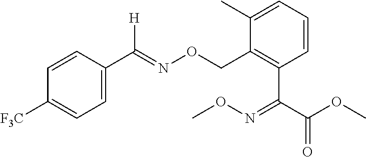 [SMILES: CO/N=C(/C(=O)OC)c1cccc(C)c1CO/N=C/c1ccc(C(F)(F)F)cc1] | 2.165 | 409 | A |
| 15 | 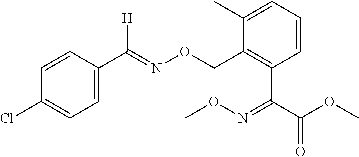 [SMILES: CO/N=C(/C(=O)OC)c1cccc(C)c1CO/N=C/c1ccc(Cl)cc1] | 2.048 | 374 | A |
| 16 | 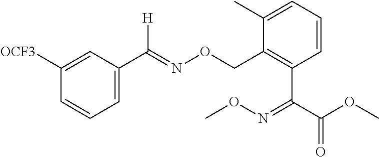 [SMILES: CO/N=C(/C(=O)OC)c1cccc(C)c1CO/N=C/c1cccc(OC(F)(F)F)c1] | 2.187 | 425 | A |
| 17 | 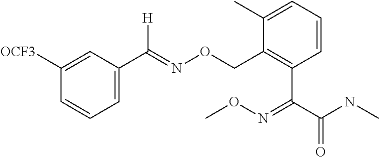 [SMILES: CNC(=O)/C(=N/OC)c1cccc(C)c1CO/N=C/c1cccc(OC(F)(F)F)c1] | 2.091 | 424 | A |
| 18 | 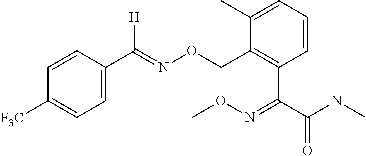 [SMILES: CNC(=O)/C(=N/OC)c1cccc(C)c1CO/N=C/c1ccc(C(F)(F)F)cc1] | 2.069 | 408 | A |
| 19 | 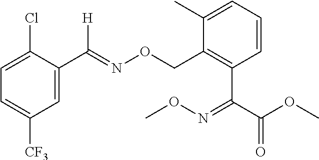 [SMILES: CO/N=C(/C(=O)OC)c1cccc(C)c1CO/N=C/c1cc(C(F)(F)F)ccc1Cl] | 2.272 | 443 | A |
| 20 | 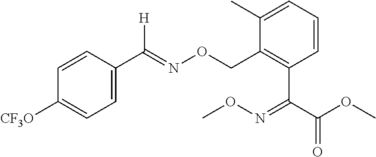 | 2.187 | 425 | A |
| 21 | 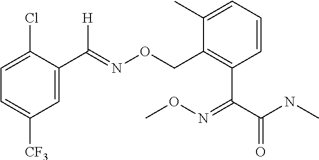 [SMILES: CNC(=O)/C(=N/OC)c1cccc(C)c1CO/N=C/c1cc(C(F)(F)F)ccc1Cl] | 2.165 | 442 | A |
| 22 | 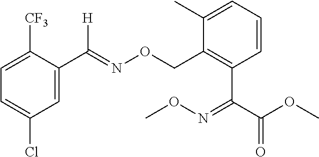 [SMILES: CO/N=C(/C(=O)OC)c1cccc(C)c1CO/N=C/c1cc(Cl)ccc1C(F)(F)F] | 2.283 | 443 | A |
| 23 | 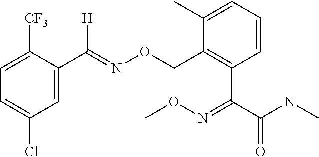 [SMILES: CNC(=O)/C(=N/OC)c1cccc(C)c1CO/N=C/c1cc(Cl)ccc1C(F)(F)F] | 2.197 | 442 | A |
| 24 | 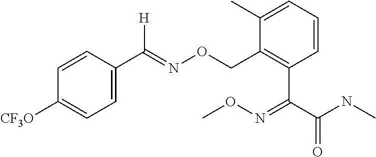 | 2.08 | 424 | A |
| 25 | 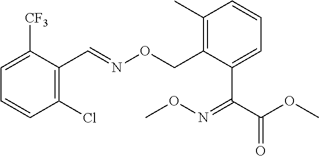 [SMILES: CO/N=C(/C(=O)OC)c1cccc(C)c1CO/N=C/c1c(Cl)cccc1C(F)(F)F] | 2.16 | 443 | A |
| 26 | 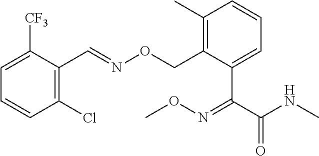 [SMILES: CNC(=O)/C(=N/OC)c1cccc(C)c1CO/N=C/c1c(Cl)cccc1C(F)(F)F] | 2.05 | 442 | A |
| 27 | 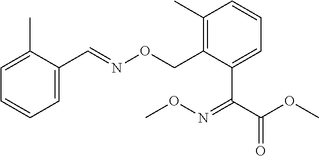 [SMILES: CO/N=C(/C(=O)OC)c1cccc(C)c1CO/N=C/c1ccccc1C] | 2.13 | 355 | A |
| 28 | 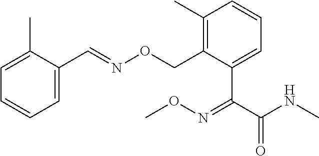 [SMILES: CNC(=O)/C(=N/OC)c1cccc(C)c1CO/N=C/c1ccccc1C] | 2.01 | 354 | A |

TABLE S2

| No. | Structure | ¹H NMR (400 MHz, CHCl₃-d) [δ] |
|---|---|---|
| 29 | 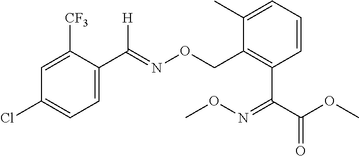 [SMILES: CO/N=C(/C(=O)OC)c1cccc(C)c1CO/N=C/c1ccc(Cl)cc1C(F)(F)F] | 8.29 (d, J = 1.9 Hz, 1H), 7.98 (d, J = 8.5 Hz, 1H), 7.64 (d, J = 1.8 Hz, 1H), 7.52-7.48 (m, 1H), 7.32 (td, J = 7.2, 14.2 Hz, 2H), 7.04 (d, J = 7.3 Hz, 1H), 5.13 (s, 2H), 4.03 (s, 3H), 3.82 (s, 3H), 2.49 (s, 3H) |
| 30 | 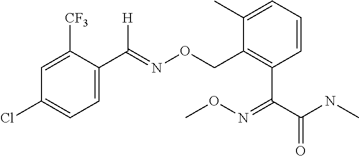 [SMILES: CNC(=O)/C(=N/OC)c1cccc(C)c1CO/N=C/c1ccc(Cl)cc1C(F)(F)F] | 8.30 (d, J = 1.6 Hz, 1H), 7.98 (d, J = 8.4 Hz, 1H), 7.63 (d, J = 1.3 Hz, 1H), 7.50 (br d, J = 8.4 Hz, 1H), 7.34-7.27 (m, 2H), 7.03 (d, J = 7.2 Hz, 1H), 6.74 (br d, J = 4.4 Hz, 1H), 5.13 (s, 2H), 3.94 (s, 3H), 2.90 (d, J = 4.9 Hz, 3H), 2.48 (s, 3H) |
| 31 | 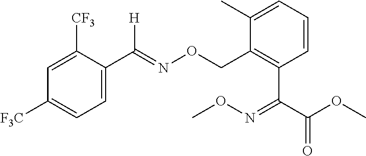 [SMILES: CO/N=C(/C(=O)OC)c1cccc(C)c1CO/N=C/c1ccc(C(F)(F)F)cc1C(F)(F)F] | 8.36 (d, J = 1.6 Hz, 1H), 8.18 (d, J = 8.3 Hz, 1H), 7.91 (s, 1H), 7.78 (d, J = 8.3 Hz, 1H), 7.37-7.29 (m, 2H), 7.04 (d, J = 7.3 Hz, 1H), 5.17 (s, 2H), 4.04 (s, 3H), 3.83 (s, 3H), 2.50 (s, 3H) |
| 32 | 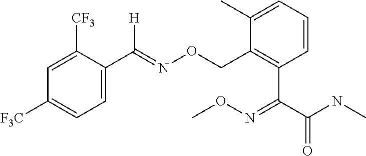 [SMILES: CNC(=O)/C(=N/OC)c1cccc(C)c1CO/N=C/c1ccc(C(F)(F)F)cc1C(F)(F)F] | 8.36 (d, J = 2.0 Hz, 1H), 8.18 (d, J = 8.3 Hz, 1H), 7.90 (s, 1H), 7.78 (d, J = 8.3 Hz, 1H), 7.35-7.28 (m, 2H), 7.04 (d, J = 7.0 Hz, 1H), 6.76 (br d, J = 4.1 Hz, 1H), 5.17 (s, 2H), 3.94 (s, 3H), 2.91 (d, J = 5.0 Hz, 3H), 2.48 (s, 3H) |
| 33 | 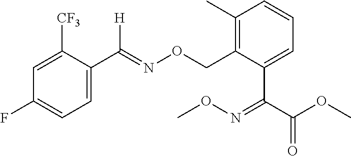 [SMILES: CO/N=C(/C(=O)OC)c1cccc(C)c1CO/N=C/c1ccc(F)cc1C(F)(F)F] | 8.30 (q, J = 2.0 Hz, 1H), 8.03 (dd, J = 5.5, 8.8 Hz, 1H), 7.38- 7.29 (m, 3H), 7.27-7.21 (m, 1H), 7.04 (dd, J = 1.3, 7.2 Hz, 1H), 5.13 (br s, 2H), 4.03-4.03 (m, 3H), 3.83-3.82 (m, 3H), 2.49 (s, 3H). |
| 34 | 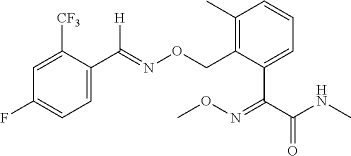 [SMILES: CNC(=O)/C(=N/OC)c1cccc(C)c1CO/N=C/c1ccc(F)cc1C(F)(F)F] | 8.31 (br d, J = 2.0 Hz, 1H), 8.04 (dd, J = 5.6, 8.7 Hz, 1H), 7.39-7.27 (m, 3H), 7.26-7.21 (m, 1H), 7.04 (d, J = 7.3 Hz, 1H), 6.75 (br d, J = 4.1 Hz, 1H), 5.12 (s, 2H), 3.94 (s, 3H), 2.90 (d, J = 5.0 Hz, 3H), 2.48 (s, 3H). |
| 35 | 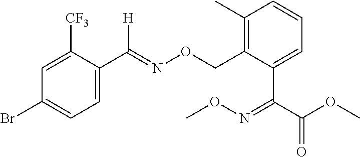 [SMILES: CO/N=C(/C(=O)OC)c1cccc(C)c1CO/N=C/c1ccc(Br)cc1C(F)(F)F] | 8.28 (d, J = 2.19 Hz, 1 H) 7.90 (d, J = 8.55 Hz, 1 H) 7.79 (d, J = 1.97 Hz, 1 H) 7.66 (dd, J = 8.44, 2.08 Hz, 1 H) 7.31-7.36 (m, 1 H) 7.27-7.31 (m, 1 H) 7.03 (dd, J = 7.34, 1.21 Hz, 1 H) 5.13 (br s, 2 H) 4.03 (s, 3 H) 3.82 (s, 3 H) 2.49 (s, 3 H). |
| 36 | 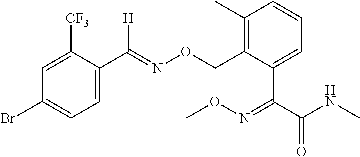 [SMILES: CNC(=O)/C(=N/OC)c1cccc(C)c1CO/N=C/c1ccc(Br)cc1C(F)(F)F] | 8.29 (d, J = 1.88 Hz, 1 H) 7.91 (d, J = 8.50 Hz, 1 H) 7.79 (d, J = 1.50 Hz, 1 H) 7.66 (br d, J = 8.50 Hz, 1 H) 7.27-7.37 (m, 2 H) 7.03 (d, J = 7.25 Hz, 1 H) 6.74 (br d, J = 4.13 Hz, 1 H) 5.13 (s, 2 H) 3.94 (s, 3 H) 2.90 (d, J = 5.00 Hz, 3 H) 2.48 (s, 3 H). |
| 37 | 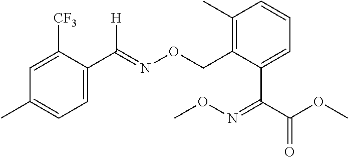 | 8.33 (d, J = 2.19 Hz, 1 H) 7.91 (d, J = 7.89 Hz, 1 H) 7.45 (s, 1 H) 7.27-7.36 (m, 3 H) 7.03 (dd, J = 7.34, 1.43 Hz, 1 H) 5.12 (s, 2 H) 4.03 (s, 3 H) 3.82 (s, 3 H) 2.50 (s, 3 H) 2.41 (s, 3 H). |
| 38 | 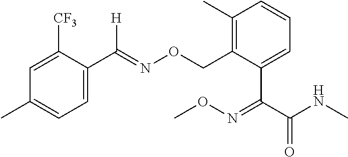 | 8.34 (d, J = 2.13 Hz, 1 H) 7.90 (d, J = 8.00 Hz, 1 H) 7.45 (s, 1 H) 7.29-7.36 (m, 2 H) 7.27 (s, 1 H) 7.00-7.08 (m, 1 H) 6.72 (br d, J = 4.25 Hz, 1 H) 5.13 (s, 2 H) 3.94 (s, 3 H) 2.89 (d, J = 5.00 Hz, 3 H) 2.48 (s, 3 H) 2.41 (s, 3 H). |

### Biological Studies

### Green House

The compound was dissolved in a mixture of acetone and/or dimethylsulfoxide and the wetting agent/emulsifier Wettol, which is based on ethoxylated alkylphenoles, in a ratio (volume) solvent-emulsifier of 99 to 1 to give a total volume of 5 ml. Subsequently, water was added to total volume of 100 ml. This stock solution was then diluted with the described solvent-emulsifier-water mixture to the final concentration given in the table below.

### Use Example 1. Protective Control of Soybean Rust on Soybeans Caused by Phakopsora pachyrhizi (PHAKPA P2)

Leaves of potted soybean seedlings were sprayed to run-off with the previously described spray solution, containing the concentration of active ingredient or their mixture as described below. The plants were allowed to air-dry. The trial plants were cultivated for 2 days in a greenhouse chamber at 23-27° C. and a relative humidity between 60 and 80%. Then the plants were inoculated with spores of Phakopsora pachyrhizi. The strain used contains the amino acid substitution F129L in the mitochondrial cytochrome b protein conferring resistance to Qo inhibitors. To ensure the success the artificial inoculation, the plants were transferred to a humid chamber with a relative humidity of about 95% and 20 to 24° C. for 24 hr. The trial plants were cultivated for up to 14 days in a greenhouse chamber at 23 to 27° C. and a relative humidity between 60 and 80%. The extent of fungal attack on the leaves was visually assessed as % diseased leaf area, the disease level of untreated controls was usually higher than 85%.

### Use Example 2. Protective Control of Soybean Rust on Soybeans Caused by Phakopsora pachyrhizi (PHAKPA P6)

Leaves of potted soybean seedlings were sprayed to run-off with the previously described spray solution, containing the concentration of active ingredient as described below. The plants were allowed to air-dry. The trial plants were cultivated for six days in a greenhouse chamber at 23-27° C. and a relative humidity between 60 and 80%. Then the plants were inoculated with spores of Phakopsora pachyrhizi. The strain used contains the amino acid substitution F129L in the mitochondrial cytochrome b protein conferring resistance to Qo inhibitors. To ensure the success the artificial inoculation, the plants were transferred to a humid chamber with a relative humidity of about 95% and 23 to 27° C. for 24 hr. The trial plants were cultivated for up to 14 days in a greenhouse chamber at 23 to 27° C. and a relative humidity between 60 and 80%. The extent of fungal attack on the leaves was visually assessed as % diseased leaf area, the disease level of untreated controls was usually higher than 85%.

The results of the abovementioned use examples are given in the following Tables. All test results below are given for the control of phytopathogenic fungi containing the amino acid substitution F129L in the mitochondrial cytochrome b protein conferring resistance to Qo inhibitors.

TABLE 1

| | | % PHAKPA Disease level | | | |
|---|---|---|---|---|---|
| Treatment with | | P2 at | P2 at | P6 at | P6 at |
| No. | Structure | 4 ppm | 16 ppm | 4 ppm | 16 ppm |
| 1 | 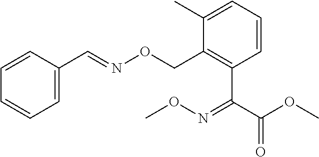 [SMILES: CO/N=C(/C(=O)OC)c1cccc(C)c1CO/N=C/c1ccccc1] | 100 | 96 | 90 | 83 |
| 2 | 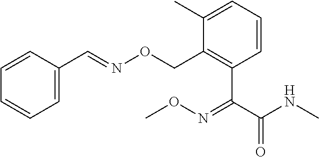 [SMILES: CNC(=O)/C(=N/OC)c1cccc(C)c1CO/N=C/c1ccccc1] | 50 | 2 | 63 | 15 |
| 3 | 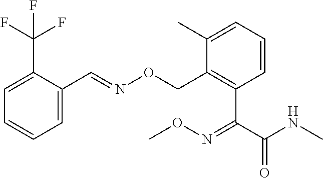 [SMILES: CNC(=O)/C(=N/OC)c1cccc(C)c1CO/N=C/c1ccccc1C(F)(F)F] | 36 | 2 | 83 | 21 |

1. A compound of formula I 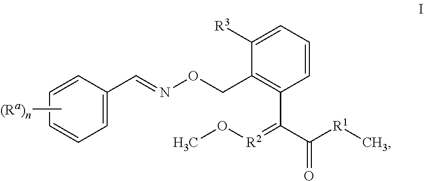
wherein
R¹ is selected from O and NH;
R² is selected from CH and N;
R³ is selected from halogen, CN, C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, —O—C₁-C₄-alkyl, —O—C₁-C₄-haloalkyl, —O—C₃-C₆-cycloalkyl, —C₁-C₂-alkyl-C₃-C₆-cycloalkyl, phenyl, 3- to 6-membered heterocycloalkyl and 5- or 6-membered heteroaryl,
wherein said heterocycloalkyl and heteroaryl besides carbon atoms contain 1, 2 or 3 heteroatoms selected from N, O and S, provided that such heterocycloalkyl and heteroaryl cannot contain 2 contiguous atoms selected from O and S,
wherein said phenyl, heterocycloalkyl and heteroaryl are bound directly or via an oxygen atom or via a C₁-C₂-alkylene linker, and wherein said phenyl and heteroaryl are unsubstituted or substituted by 1, 2 or 3 identical or different substituents selected from halogen, CN, NH₂, NO₂, C₁-C₄-alkyl, C₁-C₄-haloalkyl, —O—C₁-C₄-alkyl and —O—C₁-C₄-haloalkyl;
R^{a} is selected from halogen, CN, —NR⁵R⁶, C₁-C₄-alkyl, C₂-C₄-alkenyl, C₂-C₄-alkynyl, —O—C₁-C₄-alkyl, —C(═N—O—C₁-C₄-alkyl)-C₁-C₄-alkyl, —C(═O)—C₁-C₄-alkyl, —O—CH₂-C(═N—O—C₁-C₄-alkyl)-C₁-C₄-alkyl, C₃-C₆-cycloalkyl, C₃-C₆-cycloalkenyl, —C₁-C₂-alkyl-C₃-C₆-cycloalkyl, —O—C₃-C₆-cycloalkyl, phenyl, 3- to 6-membered heterocycloalkyl, 3- to 6-membered heterocycloalkenyl and 5- or 6-membered heteroaryl,
wherein said heterocycloalkyl, heterocycloalkenyl and heteroaryl besides carbon atoms contain 1, 2 or 3 heteroatoms selected from N, O and S, provided that such heterocycloalkyl, heterocycloalkenyl and heteroaryl cannot
contain 2 contiguous atoms selected from O and S,
wherein said phenyl, heterocycloalkyl, heterocycloalkenyl and heteroaryl are bound directly or via an oxygen atom or via a C₁-C₂-alkylene linker, and wherein the aliphatic and cyclic moieties of R^{a} are unsubstituted or carry 1, 2, 3, 4 or up to the maximum number of identical or different groups R^{b}:
R^{b} is selected from halogen, CN, NH₂, NO₂, C₁-C₄-alkyl, C₁-C₄-haloalkyl, —O—C₁-C₄-alkyl and —O—C₁-C₄-haloalkyl;
R⁵, R⁶ are independently of each other selected from the group consisting of H, C₁-C₆-alkyl, C₁-C₆-haloalkyl and C₂-C₄-alkynyl;
n is an integer selected from 0, 1, 2, 3, 4 and 5;
and in form of stereoisomers and tautomers thereof, and the N-oxides and the agriculturally acceptable salts thereof.

2. The compound according to claim 1, wherein in R¹ is selected from O and NH; and R² is selected from CH and N, provided that R² is N in case R¹ is NH.

3. The compound according to claim 2, wherein R² is N.

4. The compound according to claim 1, wherein R³ is selected from CN, halogen, C₁-C₂-alkyl, C₁-C₂-haloalkyl, C₃-C₄-cycloalkyl, —O—C₁-C₂-alkyl and —O—C₁-C₂-halalkyl.

5. The compound according to claim 4, wherein R³ is selected from the group consisting of halogen, C₁-C₂-alkyl, and C₁-C₂-haloalkyl.

6. The compound according to claim 1, wherein n is 0, 1 or 2.

7. The compound according to claim 1, wherein R^{a} is selected from the group consisting of C₁-C₃-alkyl, C₂-C₃-alkenyl, C₂-C₃-alkynyl, —O—C₁-C₃-alkyl, —C(═N—O—C₁-C₂-alkyl)-C₁-C₂-alkyl, —O—CH₂—C(═N—O—C₁-C₂-alkyl)-C₁-C₂-alkyl, C₃-C₄-cycloalkyl, —C₁-C₂-alkyl-C₃-C₄-cycloalkyl, —O—C₃-C₄-cycloalkyl, phenyl, 3- to 5-membered heterocycloalkyl and 5- or 6-membered heteroaryl, wherein said heterocycloalkyl and heteroaryl besides carbon atoms contain 1 or 2 heteroatoms selected from N, O and S, provided that such heterocycloalkyl and heteroaryl cannot contain 2 contiguous atoms selected from O and S, wherein said phenyl and heteroaryl are bound directly or via an oxygen atom or via a methylene linker, and wherein the aliphatic and cyclic moieties of R^{a} are unsubstituted or carry 1, 2 or 3 of identical or different groups R^{b} which independently of one another are selected from halogen, CN, methyl and C₁-haloalkyl.

8. An agrochemical composition comprising an auxiliary and at least one compound of formula I as defined in claim 1 or in the form of a stereoisomer and tautomer thereof or an agriculturally acceptable salt or N-oxide thereof.

9. (canceled)

10. (canceled)

11. (canceled)

12. A method for combating phytopathogenic fungi comprising:
treating curatively and/or preventively a plant or plant propagation material of said plant that is at risk of being diseased from the said phytopathogenic fungi, and/or applying to the said phytopathogenic fungi, at least one compound of formula I as defined in claim 1.

13. The method according to claim 12, wherein the phytopathogenic fungi contain an amino acid substitution F129L in the mitochondrial cytochrome b protein conferring resistance to Qo inhibitors.

14. The method according to claim 12, wherein the phytopathogenic fungi are selected from Phakopsora pachyrhizi and P. meibomiae.
